(19) United States
(12) Patent Application Publication
(10) Pub. No.: US 2005/0107409 A1
(43) Pub. Date: May 19, 2005

(54) Aromatic bicyclic compounds, preparation thereof and their use as pharmaceutical compositions

(75) Inventors: Henning Priepke, Warthausen (DE); Uwe Ries, Biberach (DE); Herbert Nar, Ochsenhausen (DE); Kai Gerlach, Ulm (DE); Roland Pfau, Biberach (DE); Georg Dahmann, Attenweiler (DE); Wolfgang Wienen, Biberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 10/784,632

(22) Filed: Feb. 23, 2004

Related U.S. Application Data
(60) Provisional application No. 60/462,487, filed on Apr. 11, 2003.

(30) Foreign Application Priority Data
Mar. 10, 2003 (DE) 10310278.7

(51) Int. Cl. A61K 31/517; A61K 31/47; C07D 239/95

(52) U.S. Cl. 514/266.4; 514/313; 544/292; 546/159

(57) ABSTRACT

The present invention relates to new aromatic bicyclic compounds of general formula 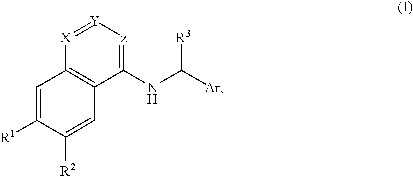
wherein Ar, X, Y and Z and R¹ to R³ are defined as in claim 1, the tautomers, the enantiomers, the diastereomers, the mixtures thereof, the prodrugs thereof and the salts thereof, as well as their preparation and their use as pharmaceutical compositions. The compounds of the above general formula I have valuable pharmacological properties, particularly an antithrombotic activity and a factor Xa-inhibiting activity.

The present invention relates to new aromatic bicyclic compounds of general formula 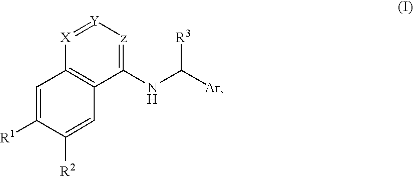
the tautomers, the enantiomers, the diastereomers, the mixtures thereof, the prodrugs thereof and the salts thereof, particularly the physiologically acceptable salts thereof with inorganic or organic acids or bases, which have valuable properties.

The compounds of the above general formula I, wherein Ar is substituted by a cyano group, are valuable intermediate products for preparing the other compounds of general formula I, and the compounds of the above general formula I which do not contain a cyano group, as well as the tautomers, the enantiomers, the diastereomers, the mixtures thereof and the salts thereof, particularly the physiologically acceptable salts thereof with inorganic or organic acids or bases, have valuable pharmacological properties, particularly an antithrombotic activity and a factor Xa-inhibiting activity.

The present application thus relates to the new compounds of the above general formula I, the preparation thereof, the pharmaceutical compositions containing the pharmacologically effective compounds, their preparation and their use.

In the above general formula
X denotes a nitrogen atom or a methyne group,
Y denotes a methyne group optionally substituted by a C₁₋₃-alkyl or amino group, or a nitrogen atom,
Z denotes a nitrogen atom or a methyne group,
R¹ denotes an amino, C₁₋₅-alkylamino, C₃₋₇-cycloalkylamino or phenyl-C₁₋₃-alkylamino group which may be substituted in each case at the amino nitrogen atom by a phenylcarbonyl or phenylsulphonyl group or by a C₁₋₃-alkyl or C₁₋₃-alkyl-carbonyl group optionally substituted in the alkyl moiety by a carboxy group, a group which may be converted in vivo into a carboxy group, an amino, C₁₋₃-alkylamino or di-(C₁₋₃-alkyl)-amino group,
a di-(C₁₋₅-alkyl)amino or N-(C₃₋₇-cycloalkyl)-C₁₋₅-alkylamino group, wherein the C₁₋₅-alkyl moiety with the exception of the 1-position may be substituted in each case by a hydroxy, C₁₋₃-alkoxy, amino, C₁₋₃-alkyl-amino or di-(C₁₋₃-alkyl)-amino group,
a 4- to 7-membered cycloalkyleneimino group, while a methylene group which is not directly adjacent to the imino group, may be substituted by a hydroxy, C₁₋₃-alkoxy, amino, C₁₋₃-alkyl-amino or di-(C₁₋₃-alkyl)-amino group,
a 4- to 7-membered cycloalkyleneiminocarbonyl or cycloalkyleneiminosulphonyl group, while
   the cycloalkyleneimino moiety may be substituted by a C₁₋₃-alkyl, amino-C₁₋₃-alkyl, C₁₋₃-alkylamino-C₁₋₃-alkyl, di-(C₁₋₃-alkyl)-amino-C₁₋₃-alkyl, amingocarbonyl, C₁₋₃-alkylamino-carbonyl, N-(C₃₋₇-cycloalkyl)-C₁₋₅-alkylaminocarbonyl, N-(phenyl-C₁₋₃-alkyl)-C₁₋₅-alkylaminocarbonyl or di-(C₁₋₃-alkyl)-aminocarbonyl group or
   a methylene group not adjacent to the imino group may be substituted by a hydroxy, benzyloxy, amino, C₁₋₃-alkylamino or di-(C₁₋₃-alkyl)-amino group and/or
   a methylene group in the 3 position of a 5-, 6- or 7-membered cycloalkyleneimino group may be replaced by a sulphur atom or by a sulphinyl or sulphonyl group or
   a methylene group in the 4 position of a 6- or 7-membered cycloalkyleneimino group may be replaced by an oxygen or sulphur atom or by a —NH—, —N(C₂₋₃-alkanoyl)-, sulphinyl or sulphonyl group and/or
   a —CH₂—CH₂— group in a 5- to 7-membered cycloalkyleneimino group may be replaced by a —NH—CO— group,
a 2,5-dihydropyrrol-1-yl-carbonyl or 1,2,5,6-tetrahydropyridin1-yl-carbonyl group optionally substituted by a C₁₋₃-alkyl, amino-C₁₋₃-alkyl, C₁₋₃-alkylamino-C₁₋₃-alkyl, di-(C₁₋₃-alkyl)-amino-C₁₋₃-alkyl, aminocarbonyl, C₁₋₃-alkylamino-carbonyl, N—(C₃₋₇-cycloalkyl)-C₁₋₅-alkylaminocarbonyl, N-(phenyl-C₁₋₃-alkyl)-C₁₋₅-alkylaminocarbonyl or di-(C₁₋₃-alkyl)-aminocarbonyl group,
an aminosulphonyl or aminocarbonyl group optionally substituted by one or two C₁₋₃-alkyl, phenyl-C₁₋₃-alkyl or C₃₋₇-cycloalkyl groups, while the substituents may be identical or different,
a straight-chain or branched C₁₋₅-alkylcarbonyl group,
a C₃₋₇-cycloalkyl-carbonyl group, while
   the methylene group in the 3 or 4 position in a C₅₋₇-cycloalkyl-carbonyl group may be replaced by a —NH— group, while
   the hydrogen atom of the —NH— group may be replaced by a C₁₋₃-alkyl, C₁₋₃-alkyl-carbonyl, phenylcarbonyl or phenylsulphonyl group,
a phenylcarbonyl or heteroarylcarbonyl group or
a C₁₋₃-alkyl group optionally substituted by an amino, C₁₋₃-alkylamino, di-(C₁₋₃-alkyl)-amino, hydroxy, phenyl or a 4- to 7-membered cycloalkyleneimino group, while
   the phenyl substituents may be substituted by a fluorine, chlorine or bromine atom, by a trifluoromethyl, C₁₋₃-alkyl or C₁₋₃-alkoxy group,
R² denotes a hydrogen, fluorine, chlorine or bromine atom,
a C₁₋₃-alkyl group wherein the hydrogen atoms may be wholly or partly replaced by fluorine atoms, or
a C₂₋₃-alkenyl, C₂₋₃-alkynyl, hydroxy, C₁₋₃-alkoxy or trifluoromethoxy group,
R³ denotes a hydrogen atom,
a straight-chain or branched C₁₋₆-alkyl group which is optionally substituted by a hydroxy, carboxy, C₁₋₃-alkoxy-carbonyl, amino, C₁₋₃-alkylamino, di-(C₁₋₃-alkyl)-amino, C₁₋₃-alkyl-carbonylamino, C₁₋₅-alkoxy-carbonylamino or phenyl-C₁₋₃-alkoxy-carbonylamino group,
a methyl or ethyl group substituted in each case
   by a phenyl or heteroaryl group which are substituted optionally in each case by a hydroxy, C₁₋₄-alkyloxy, benzyloxy, hydroxycarbonyl-C₁₋₃-alkoxy, C₁₋₃-alkyloxy-carbonyl-C₁₋₃-alkyloxy, aminocarbonyl-C₁₋₃-alkyloxy, C₁₋₃-alkylaminocarbonyl-C₁₋₃-alkyloxy, di-(C₁₋₃-alkyl)-aminocarbonyl-C₁₋₃-alkyloxy, carboxy, C₁₋₃-alkyloxy-carbonyl group,
   by a 1-H-pyridonyl or 1-(C₁₋₃-alkyl)-pyridonyl group,
   by a 4- to 7-membered cycloalkyleneimino group or
   by a 4- to 7-membered cycloalkyl group wherein one or two methylene groups separated from one another by at least a methylene group are each replaced by an oxygen or sulphur atom or by a —NH— or —N(C₁₋₃-alkyl)-group and wherein, if the cycloalkyl group contains an —NH— or an —N(C₁₋₃-alkyl)- group, a methylene group adjacent to the nitrogen atom and, if the cycloalkyl group contains a total of two —NH— or —N(C₁₋₃-alkyl)- groups, a methylene group adjacent to both nitrogen atoms may be replaced by a carbonyl group, or
a phenyl or heteroaryl group which may be substituted in each case by a hydroxy, C₁₋₄-alkyloxy, benzyloxy, hydroxycarbonyl-C₁₋₃-alkoxy, C₁₋₃-alkyloxy-carbonyl-C₁₋₃-alkyloxy, aminocarbonyl-C₁₋₃-alkyloxy, C₁₋₃-alkylaminocarbonyl-C₁₋₃-alkyloxy, di-(C₁₋₃-alkyl)-aminocarbonyl-C₁₋₃-alkyloxy, carboxy, C₁₋₃-alkyloxy-carbonyl group, and
Ar denotes a phenyl group substituted by the groups R⁴, R⁵ and R⁶, where
   R⁴ denotes a cyano group,
   an amidino group optionally substituted by one or two hydroxy, C₁₋₃-alkyl, C₁₋₈-alkyl-carbonyl, C₁₋₈-alkoxy-carbonyl or benzoyl groups,
   an amino-C₁₋₃-alkyl, C₁₋₃-alkylamino-C₁₋₃-alkyl or di-(C₁₋₃-alkyl)amino-C₁₋₃-alkyl group or
   a group of formula 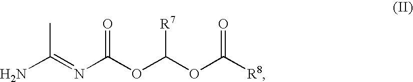
   wherein R⁷ denotes a hydrogen atom or a C₁₋₃-alkyl group and
   R⁸ denotes a C₁₋₃-alkyl group,
   R⁵ denotes a hydrogen, fluorine, chlorine or bromine atom or a trifluoromethyl, C₁₋₃-alkyl, hydroxy, hydroxy-C₁₋₃-alkyl, C₁₋₃-alkoxy, benzyloxy, C₁₋₃-alkoxy-C₁₋₃-alkyl, amino, C₁₋₃-alkylamino or di-(C₁₋₃-alkyl)amino group and
   R⁶ denotes a hydrogen, fluorine, chlorine or bromine atom or a C₁₋₃-alkyl group,
or a thienylene, thiazolylene, pyridinylene, pyrimidinylene, pyrazinylene or pyridazinylene group optionally substituted in the carbon skeleton by the groups R⁴ and R⁵, where R⁴ and R⁵ are as hereinbefore defined,
while, unless otherwise mentioned, the expression a “heteroaryl group” refers to a monocyclic 5- or 6-membered heteroaryl group optionally substituted in the carbon skeleton by a C₁₋₃-alkyl, carboxy, C₁₋₃-alkoxy-carbonyl or C₁₋₃-alkoxy-carbonylamino group, while
   the 6-membered heteroaryl group contains one, two or three nitrogen atoms and
   the 5-membered heteroaryl group denotes an imino group optionally substituted by a C₁₋₃-alkyl or phenyl-C₁₋₃-alkyl group, an oxygen or sulphur atom or
   an imino group optionally substituted by a C₁₋₃-alkyl, amino-C₁₋₃-alkyl, C₁₋₃-alkylamino-C₁₋₃-alkyl, di-(C₁₋₃-alkyl)-amino-C₁₋₃-alkyl or phenyl-C₁₋₃-alkyl group or an oxygen or sulphur atom and additionally a nitrogen atom or
   an imino group optionally substituted by a C₁₋₃-alkyl or phenyl-C₁₋₃-alkyl group and two or three nitrogen atoms,
   and moreover a phenyl ring may be fused to the above-mentioned monocyclic heteroaryl groups via two adjacent carbon atoms
   and the bond is effected via a nitrogen atom or a carbon atom of the heterocyclic moiety or a fused-on phenyl ring,
while the alkyl and alkoxy groups contained in the definitions which have more than two carbon atoms may, unless otherwise stated, be straight-chain or branched,
and the hydrogen atoms of the methyl or ethyl groups contained in the definitions may be wholly or partly replaced by fluorine atoms.

By a group which may be converted in vivo into a carboxy group is meant for example a hydroxymethyl group, a carboxy group esterified with an alcohol wherein the alcoholic moiety preferably denotes a C₁₋₆-alkanol, a phenyl-C₁₋₃-alkanol, a C₃₋₉-cycloalkanol, while a C₅₋₈-cycloalkanol may additionally be substituted by one or two C₁₋₃-alkyl groups, a C₅₋₈-cycloalkanol wherein a methylene group in the 3 or 4 position is replaced by an oxygen atom or by an imino group optionally substituted by a C₁₋₃-alkyl, phenyl-C₁₋₃-alkyl, phenyl-C₁₋₃-alkoxycarbonyl or C₂₋₆-alkanoyl group and the cycloalkanol moiety may additionally be substituted by one or two C₁₋₃-alkyl groups, a C₄₋₇-cycloalkenol, a C₃₋₅-alkenol, a phenyl-C₃₋₅-alkenol, a C₃₋₅-alkynol or phenyl-C₃₋₅-alkynol, with the proviso that no bond to the oxygen atom starts from a carbon atom which carries a double or triple bond, a C₃₋₈-cycloalkyl-C₁₋₃-alkanol, a bicycloalkanol with a total of 8 to 10 carbon atoms which may additionally be substituted in the bicycloalkyl moiety by one or two C₁₋₃-alkyl groups, a 1,3-dihydro-3-oxo-1-isobenzofuranol or an alcohol of formula
Rₐ—CO—O—(R_{b}CR_{c})—OH,
wherein
Rₐ denotes a C₁₋₈-alkyl, C₅₋₇-cycloalkyl, phenyl or phenyl-C₁₋₃-alkyl group,
R_{b} denotes a hydrogen atom, a C₁₋₃-alkyl, C₅₋₇-cycloalkyl or phenyl group and
R_{c} denotes a hydrogen atom or a C₁₋₃-alkyl group.

Those compounds of general formula (I) which contain a group that can be cleaved in vivo, as well as those wherein X, Y and Z and R¹ to R³ are as hereinbefore defined and Ar is as hereinbefore defined, with the proviso that Ar contains an amidino group substituted at the nitrogen atom by one or two hydroxy, C₁₋₃-alkyl, C₁₋₈-alkyl-carbonyl, C₁₋₈-alkoxy-carbonyl or benzoyl groups or by a group of formula II, or a C₁₋₃-alkylamino-C₁₋₃-alkyl or di-(C₁₋₃-alkyl)amino-C₁₋₃-alkyl group, are prodrugs for those compounds of general formula (I) wherein Ar contains an amidino or aminoalkyl group.

Preferred compounds of general formula I are those wherein
X denotes a nitrogen atom or a methyne group,
Y denotes a methyne group optionally by substituted a C₁₋₃-alkyl group,
Z denotes a nitrogen atom or a methyne group,
R¹ denotes an amino, C₁₋₅-alkylamino or C₃₋₇-cycloalkylamino group which may be substituted in each case at the amino nitrogen atom by a C₁₋₃-alkyl or C₁₋₃-alkyl-carbonyl group optionally substituted in the alkyl moiety by a carboxy group, a group which may be converted in vivo into a carboxy group, an amino, C₁₋₃-alkylamino or di-(C₁₋₃-alkyl)-amino group,
a 4- to 7-membered cycloalkyleneiminocarbonyl or cycloalkyleneiminosulphonyl group, while
   the cycloalkyleneimino moiety may be substituted by a C₁₋₃-alkyl, amino-C₁₋₃-alkyl, C₁₋₃-alkylamino-C₁₋₃-alkyl, di-(C₁₋₃-alkyl)-amino-C₁₋₃-alkyl, aminocarbonyl, C₁₋₃-alkylamino-carbonyl, N—(C₃₋₇-cycloalkyl)-C₁₋₅-alkylaminocarbonyl or di-(C₁₋₃-alkyl)-aminocarbonyl group or
   a methylene group not adjacent to the imino group may be substituted by a hydroxy, benzyloxy, amino, C₁₋₃-alkylamino or di-(C₁₋₃-alkyl)-amino group and/or
   a methylene group in the 3 position of a 5-, 6- or 7-membered cycloalkyleneimino group may be replaced by a sulphur atom or by a sulphinyl or sulphonyl group or
   a methylene group in the 4 position of a 6- or 7-membered cycloalkyleneimino group may be replaced by an oxygen or sulphur atom or by an —NH—, —N(C₂₋₃-alkanoyl)-, sulphinyl or sulphonyl group and/or
   a —CH₂—CH₂— group in a 5- to 7-membered cycloalkyleneimino group may be replaced by a —NH—CO— group,
a 2,5-dihydropyrrol-1-yl-carbonyl or 1,2,5,6-tetrahydropyridin 1-yl-carbonyl group optionally substituted by a C₁₋₃-alkyl, amino-C₁₋₃-alkyl, C₁₋₃-alkylamino-C₁₋₃-alkyl, di-(C₁₋₃-alkyl)-amino-C₁₋₃-alkyl, aminocarbonyl, C₁₋₃-alkylamino-carbonyl, N—(C₃₋₇-cycloalkyl)-C₁₋₅-alkylaminocarbonyl, N-(phenyl-C₁₋₃-alkyl)-C₁₋₅-alkylaminocarbonyl or di-(C₁₋₃-alkyl)-aminocarbonyl group,
an aminosulphonyl or aminocarbonyl group optionally substituted by one or two C₁₋₃-alkyl, phenyl-C₁₋₃-alkyl or C₃₋₇-cycloalkyl groups, while the substituents may be identical or different,
a straight-chain or branched C₁₋₅-alkylcarbonyl group or
a C₃₋₇-cycloalkyl-carbonyl group,
R² denotes a hydrogen, fluorine, chlorine or bromine atom,
a C₁₋₃-alkyl group wherein the hydrogen atoms may be wholly or partly replaced by fluorine atoms, or
a C₂₋₃-alkenyl, C₂₋₃-alkynyl, hydroxy, C₁₋₃-alkoxy or trifluoromethoxy group,
R³ denotes a hydrogen atom,
a straight-chain or branched C₁₋₆-alkyl group which is optionally substituted by a hydroxy, carboxy, C₁₋₃-alkoxy-carbonyl, amino, C₁₋₃-alkylamino, di-(C₁₋₃-alkyl)-amino, C₁₋₃-alkyl-carbonylamino, C₁₋₅-alkoxy-carbonylamino or phenyl-C₁₋₃-alkoxy-carbonylamino group, or
a methyl group which is substituted
   by a phenyl or heteroaryl group which are optionally substituted in each case by a hydroxy, C₁₋₄-alkyloxy, benzyloxy, hydroxycarbonyl-C₁₋₃-alkoxy, C₁₋₃-alkyloxy-carbonyl-C₁₋₃-alkyloxy, aminocarbonyl-C₁₋₃-alkyloxy, C₁₋₃-alkylaminocarbonyl-C₁₋₃-alkyloxy, di-(C₁₋₃-alkyl)-aminocarbonyl-C₁₋₃-alkyloxy, carboxy, C₁₋₃-alkyloxy-carbonyl group, and
Ar denotes a phenyl group substituted by the groups R⁴, R⁵ and R⁶, while
   R⁴ denotes a cyano group,
   an amidino group optionally substituted by one or two hydroxy, C₁₋₃-alkyl, C₁₋₈-alkyl-carbonyl, C₁₋₈-alkoxy-carbonyl or benzoyl groups,
   an amino-C₁₋₃-alkyl, C₁₋₃-alkylamino-C₁₋₃-alkyl or di-(C₁₋₃-alkyl)amino-C₁₋₃-alkyl group or
   a group of formula 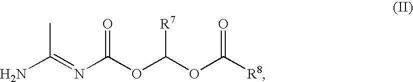
   wherein R⁷ denotes a hydrogen atom or a C₁₋₃-alkyl group and
   R⁸ denotes a C₁₋₃-alkyl group,
   R⁵ denotes a hydrogen, fluorine, chlorine or bromine atom or a trifluoromethyl, C₁₋₃-alkyl, hydroxy, benzyloxy, amino or C₁₋₃-alkylamino group and
   R⁶ denotes a hydrogen, chlorine or bromine atom or a C₁₋₃-alkyl group,
or a thienylene, thiazolylene, pyridinylene, pyrimidinylene, pyrazinylene or pyridazinylene group optionally substituted in the carbon skeleton by the groups R⁴ and R⁵, while R₄ and R₅ are as hereinbefore defined,
while, unless otherwise mentioned, by the term a “heteroaryl group” is meant a monocyclic 5- or 6-membered heteroaryl group optionally substituted in the carbon skeleton by a C₁₋₃-alkyl, carboxy, C₁₋₃-alkoxy-carbonyl or C₁₋₃-alkoxy-carbonylamino group, while
   the 6-membered heteroaryl group contains one, two or three nitrogen atoms and
   the 5-membered heteroaryl group contains an imino group optionally substituted by a C₁₋₃-alkyl or phenyl-C₁₋₃-alkyl group, or an oxygen or sulphur atom or
   an imino group optionally substituted by a C₁₋₃-alkyl, amino-C₁₋₃-alkyl, C₁₋₃-alkylamino-C₁₋₃-alkyl, di-(C₁₋₃-alkyl)-amino-C₁₋₃-alkyl or phenyl-C₁₋₃-alkyl group, or an oxygen or sulphur atom and additionally contains a nitrogen atom or
   an imino group optionally substituted by a C₁₋₃-alkyl or phenyl-C₁₋₃-alkyl group and two or three nitrogen atoms,
   and moreover a phenyl ring may be fused to the above-mentioned monocyclic heteroaryl groups via two adjacent carbon atoms
   and the bond is effected via a nitrogen atom or a carbon atom of the heterocyclic moiety or a fused-on phenyl ring,
while the alkyl and alkoxy groups contained in the definitions which have more than two carbon atoms may, unless otherwise stated, be straight-chain or branched,
and the hydrogen atoms of the methyl or ethyl groups contained in the definitions may be wholly or partly replaced by fluorine atoms,
the tautomers, the enantiomers, the diastereomers, the mixtures thereof, the prodrugs thereof and the salts thereof.

Particularly preferred compounds of general formula I are those wherein
X denotes a nitrogen atom or a methyne group,
Y denotes a methyne group and
Z denotes a nitrogen atom or a methyne group,
R¹ denotes an amino, C₁₋₅-alkylamino or C₃₋₇-cycloalkylamino group which may be substituted in each case at the amino nitrogen atom by a C₁₋₃-alkyl or C₁₋₃-alkyl-carbonyl group optionally substituted in the alkyl moiety by a carboxy group, a group which may be converted in vivo into a carboxy group, an amino, C₁₋₃-alkylamino or di-(C₁₋₃-alkyl)-amino group,
a 4- to 7-membered cycloalkyleneiminocarbonyl group, while
   the cycloalkyleneimino moiety may be substituted by a C₁₋₃-alkyl, amino-C₁₋₃-alkyl, C₁₋₃-alkylamino-C₁₋₃-alkyl, di-(C₁₋₃-alkyl)-amino-C₁₋₃-alkyl, aminocarbonyl, C₁₋₃-alkylamino-carbonyl or di-(C₁₋₃-alkyl)-aminocarbonyl group or
   a methylene group not adjacent to the imino group may be substituted by a hydroxy, benzyloxy, amino, C₁₋₃-alkylamino or di-(C₁₋₃-alkyl)-amino group and/or
   a methylene group in the 3 position of a 5-, 6- or 7-membered cycloalkyleneimino group may be replaced by a sulphur atom or by a sulphinyl or sulphonyl group or
   a methylene group in the 4 position of a 6- or 7-membered cycloalkyleneimino group may be replaced by an oxygen or sulphur atom or by an —NH—, —N(C₂₋₃-alkanoyl)-, sulphinyl or sulphonyl group and/or
   a —CH₂—CH₂— group in a 5- to 7-membered cycloalkyleneimino group may be replaced by a —NH—CO— group,
   a 2,5-dihydropyrrol-1-yl-carbonyl or 1,2,5,6-tetrahydropyridin 1-yl-carbonyl group optionally substituted by a C₁₋₃-alkyl, amino-C₁₋₃-alkyl, C₁₋₃-alkylamino-C₁₋₃-alkyl, di-(C₁₋₃-alkyl)-amino-C₁₋₃-alkyl, aminocarbonyl, C₁₋₃-alkylamino-carbonyl, N—(C₃₋₇-cycloalkyl)-C₁₋₅-alkylaminocarbonyl, N-(phenyl-C₁₋₃-alkyl)-C₁₋₅-alkylaminocarbonyl or di-(C₁₋₃-alkyl)-aminocarbonyl group,
an aminocarbonyl group optionally substituted by one or two C₁₋₃-alkyl or C₃₋₇-cycloalkyl groups, while the substituents may be identical or different,
a straight-chain or branched C₁₋₅-alkylcarbonyl group or
a C₃₋₇-cycloalkyl-carbonyl group,
R² denotes a hydrogen, fluorine, chlorine or bromine atom,
a C₁₋₃-alkyl group wherein the hydrogen atoms may be wholly or partly
replaced by fluorine atoms, or
a C₁₋₃-alkoxy or trifluoromethoxy group,
R³ denotes a hydrogen atom,
a straight-chain or branched C₁₋₆-alkyl group which is optionally substituted by a hydroxy, carboxy or C₁₋₃-alkoxy-carbonyl group, or
a methyl group which is substituted by a phenyl group, and
Ar denotes a phenyl group substituted by the groups R⁴ and R⁵, where
   R⁴ denotes a cyano group,
   an amidino group optionally substituted by one or two hydroxy, C₁₋₈-alkyl-carbonyl, C₁₋₈-alkoxy-carbonyl or benzoyl groups,
   an amino-C₁₋₃-alkyl group or
   a group of formula 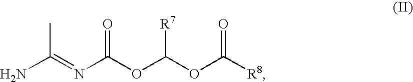
   wherein R⁷ denotes a hydrogen atom or a C₁₋₃-alkyl group and
   R⁸ denotes a C₁₋₃-alkyl group,
   and R⁵ denotes a hydrogen atom or a hydroxy group,
or a thienylene group optionally substituted in the carbon skeleton by the group R₄, where R₄ is as hereinbefore defined,
while the alkyl and alkoxy groups contained in the definitions which have more than two carbon atoms may, unless otherwise stated, be straight-chain or branched,
and the hydrogen atoms of the methyl or ethyl groups contained in the definitions may be wholly or partly replaced by fluorine atoms,
the tautomers, the enantiomers, the diastereomers, the mixtures thereof and the salts thereof.

Most particularly preferred are those compounds of general formula I wherein
X, Y, Z, R², R³ and Ar are as hereinbefore defined and
   R¹ denotes a C₃₋₇-cycloalkylamino group which is substituted at the amino nitrogen atom by a C₁₋₃-alkyl-carbonyl group,
   an azetidin-1-ylcarbonyl group optionally substituted in the 3 position by a dimethylamino group,
   a pyrrolidin-1-yl-carbonyl or piperidin-1-ylcarbonyl group optionally substituted in the 2 position by an aminomethyl or in the 3 position by an amino, aminomethyl, hydroxy or benzyloxy group,
   a 2,5-dihydropyrrol-1-yl-carbonyl or thiazolidin-3-yl-carbonyl group or
   a di-(C₁₋₃-alkyl)-aminocarbonyl or N-cyclohexyl-N—(C₁₋₃-alkyl)-amino-carbonyl group,
the tautomers, the enantiomers, the diastereomers, the mixtures thereof and the salts thereof.

Particularly preferred are the compounds with the absolute configuration shown in formula (Ia): 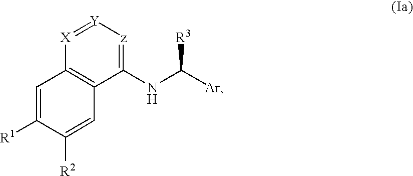
wherein X, Y, Z, R¹, R², R³ and Ar are as hereinbefore defined, the tautomers, the diastereomers, the mixtures thereof and the salts thereof.

The following compounds of the above general formula I are mentioned by way of example:
(1) 3-{[7-(2,5-dihydropyrrol-1-yl-carbonyl)-quinazolin-4-yl]aminomethyl}-4-hydroxy-benzamidine 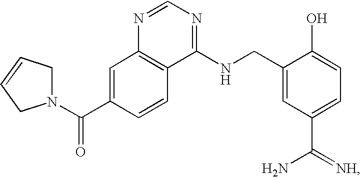
(2) 3-{2-phenyl-1-[7-(pyrrolidin-1-yl-carbonyl)-quinolin-4-ylamino]-ethyl}-benzamidine 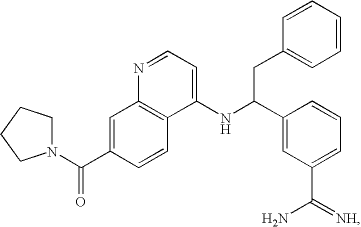
(3) 4-hydroxy-3-{[7-methoxy-6-(pyrrolidin-1-yl-carbonyl)-isoquinolin-1-yl]aminomethyl}-benzamidine 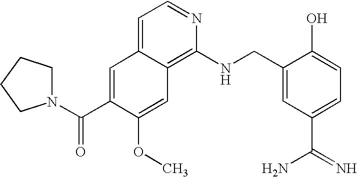
(4) 4-hydroxy-3-{2-phenyl-1-[7-(pyrrolidin-1-yl-carbonyl)-quinazolin-4-ylamino]-ethyl}-benzamidine 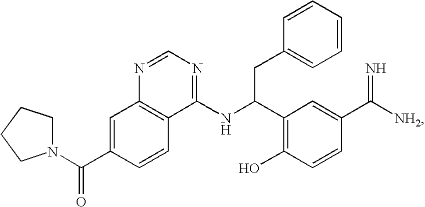
(5) 4-hydroxy-3-{[6-methyl-7-(pyrrolidin-1-yl-carbonyl)-quinazolin-4-yl]aminomethyl}-benzamidine 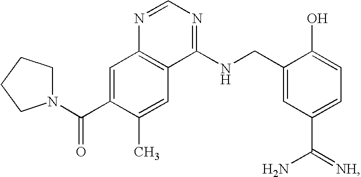
(6) ethyl 3-(3-amidino-phenyl)-3-{[6-chloro-7-(pyrrolidin-1-yl-carbonyl)-quinazolin-4-yl]amino}-propionate 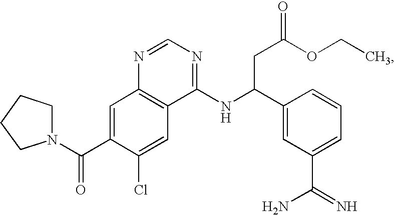
(7) 3-{[6-(N-acetyl-N-cyclopentylamino)-7-methyl-isoquinolin-1-yl]aminomethyl}-4-hydroxy-benzamidine 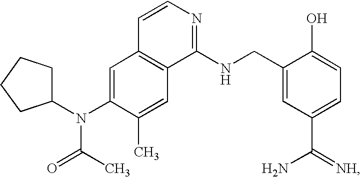
(8) N-acetoxymethoxycarbonyl-4-hydroxy-3-{[6-methyl-7-(pyrrolidin-1-yl-carbonyl)-quinazolin-4-yl]aminomethyl}-benzamidine 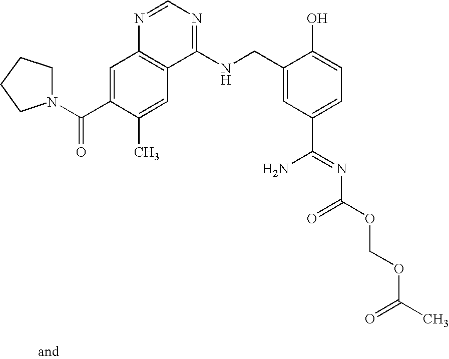
(9) 4-hydroxy-3-{[6-chloro-7-(pyrrolidin-1-yl-carbonyl)-quinazolin-4-yl]aminomethyl}-benzamidine 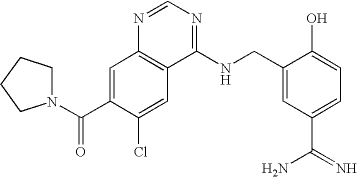
and the tautomers and the salts thereof.

According to the invention the compounds of general formula I are obtained by methods known per se, for example by the following methods:
(a) In order to prepare a compound of general formula (I) wherein Ar, X, Y, Z and R¹ to R³ are as hereinbefore defined:
coupling a compound of general formula 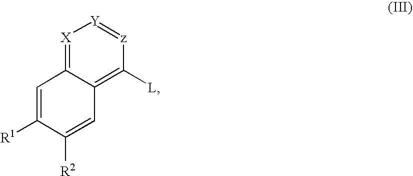
wherein
R¹, R², X, Y and Z are as hereinbefore defined and L denotes a leaving group such as a halogen atom, a sulphonyloxy or aryloxy group, e.g. a chlorine, bromine or iodine atom, a trifluoromethylsulphonyloxy, phenyloxy or p-nitrophenyloxy group,
with a compound of general formula 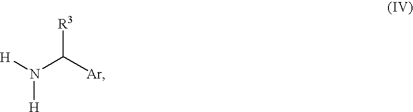
wherein Ar and R³ are as hereinbefore defined and
and, if Ar is substituted by a cyano group, optionally subsequently converting the cyano compound thus obtained into one of the optionally substituted amidino or aminoalkyl compounds mentioned previously.

The coupling reaction is conveniently carried out in a solvent such as toluene, dioxane, dimethoxyethane, dimethylformamide, dimethylsulphoxide or tetrahydrofuran, preferably in the presence of a base such as sodium-tert.-butoxide, bis-(trimethylsilyl)-lithiumamide, potassium carbonate, caesium carbonate, ethyldiisopropylamine (Hunig base) or triethylamine, at a temperature between 0° C. and 200° C., preferably between 0° C. and 150° C., optionally using a suitable catalyst, for example bis-(tri-o-tolylphosphine)-palladium-(II)-chloride, tris-(dibenzylideneacetone)-dipalladium(0)/tris-o-tolylphosphine, tris-(dibenzylideneacetone)-dipalladium(0)/tris-(2-furyl)phosphane, tris-(dibenzylideneacetone)-dipalladium(0)/2,2′-bis-(diphenylphosphino)-1,1′-binaphthyl, tetrakis-(triphenylphosphine)-palladium(0), 1,1′-bis-(diphenylphosphino)-ferrocene-palladium-dichloride or palladium-II-acetate/1,3-bis-(triphenylphosphino)-propane.

The coupling reaction may, however, also be carried out without the addition of solvent in bulk by melting the compounds of general formulae III and IV at temperatures between ambient temperature and 250° C., optionally in the presence of one of the bases previously mentioned and/or optionally using one of the catalysts mentioned previously.

The compounds of general formulae III and IV used as starting materials, some of which are known from the literature, are obtained by methods known from the literature. Their preparation is also described in the Examples.

A compound of the above general formula (III), wherein
R¹ and R² are as hereinbefore defined,
Y denotes a methyne group,
X and Z in each case denote a nitrogen atom and
L denotes a leaving group such as a halogen atom, for example a chlorine or bromine atom,
may be prepared, for example, by the following method:
cyclising a compound of general formula 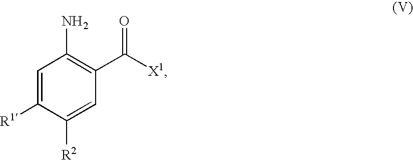
wherein
R^{1′} has one of the meanings given for R¹ hereinbefore or denotes an optionally protected carboxy group, which can then be converted into the groups defined for R¹ hereinbefore,
R² is as hereinbefore defined and
X¹ denotes a hydroxy or C₁₋₄-alkoxy group or a halogen atom,
with formamide and subsequently reacting the resulting compound of general formula 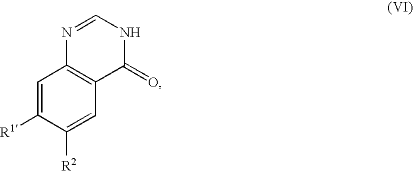
wherein R^{1′} and R² are as hereinbefore defined, with a halogenating agent, for example with thionyl chloride, thionyl bromide or oxalyl chloride.

The cyclisation is carried out for example in a high-boiling solvent such as chlorobenzene, xylene, dimethylformamide, dimethylsulphoxide or sulpholane or also without any further solvent in the presence of excess formamide at temperatures between 100 and 200° C., preferably between 130 and 170° C.

The subsequent reaction with a halogenating agent, for example with thionyl chloride, thionyl bromide or oxalyl chloride, is conveniently carried out either in bulk, without solvent, in the presence of dimethylformamide as catalyst or by the addition of a solvent such as dimethylformamide, pyridine, 4-(N,N-dimethylamino)-pyridine, benzene, carbon tetrachloride, 1,2-dichloroethane or chloroform at temperatures between 20 and 120° C.

A compound of the above general formula (III) wherein
R¹ and R² are as hereinbefore defined,
X and Y in each case denote a methyne group,
Z denotes a nitrogen atom and
L denotes a leaving group such as a halogen atom, for example a chlorine or bromine atom,
may be prepared by the following process, for example:
reacting a compound of general formula 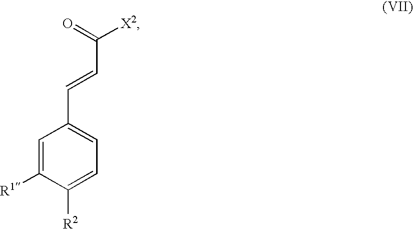
wherein
R^{1″} is as hereinbefore defined or denotes a bromine atom,
R² is as hereinbefore defined and
X² denotes a hydroxy, C₁₋₆-alkoxycarbonyloxy or C₁₋₄-alkoxy group or a halogen atom, with sodium azide,
subsequently cyclising the resulting compound to form a compound of general formula 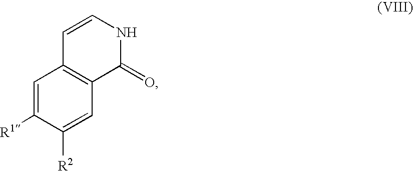
wherein R^{1″} is as hereinbefore defined or denotes a bromine atom and R² is as hereinbefore defined,
and subsequently reacting with a halogenating agent, for example with thionyl chloride, thionyl bromide or oxalyl chloride.

If R^{1″} denotes a bromine atom it can be converted into the corresponding carboxylic acid by treatment with n-butyllithium and scavenging the intermediate product with carbon dioxide.

The reaction with sodium azide is carried out for example in acetone in the presence of triethylamine and ethyl chloroformate at temperatures between 0° C. and ambient temperature.

The rearrangement-cyclisation sequence is carried out for example in a high-boiling solvent such as diphenylether, chlorobenzene, xylene, dimethylformamide, dimethylsulphoxide, sulpholane or also without any additional solvent in the presence of tributylamine at temperatures between 100 and 250° C., preferably between 200 and 250° C.

The subsequent reaction with a halogenating agent may be carried out for example as described above.

The group R¹ as hereinbefore defined may, however, also be introduced only after the cyclisation to obtain the isoquinoline by substitution of a halogen atom and other modifications, as described above (see Diagram 1): 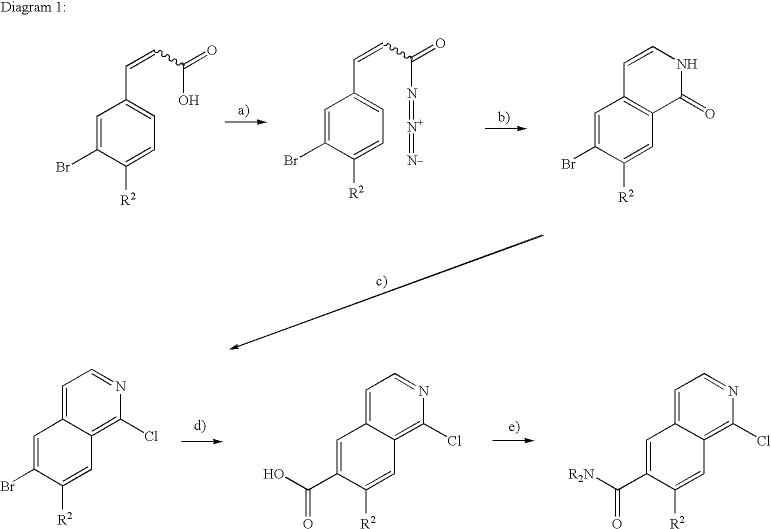
a) see synthesis of VII (for X=Azide); b) see synthesis of VIII; c) reaction to form the 1-haloisoquinoline as described above; d) nBuLi, −70° C., THF, CO₂; e) R₂NH, N-methylmorpholine, TBTU, DMF, or by other amide coupling processes;
Nitrogen-bonded groups R¹ may for example also be synthesised by palladium-catalysed Buchwald couplings (J. P. Wolfe et. al. Acc. Chem. Res. 1998, 31, 805 and J. F. Hartwig, Angew. Chem. Int. Ed. 1998, 37, 2046) according to the following diagram (Diagram 2): 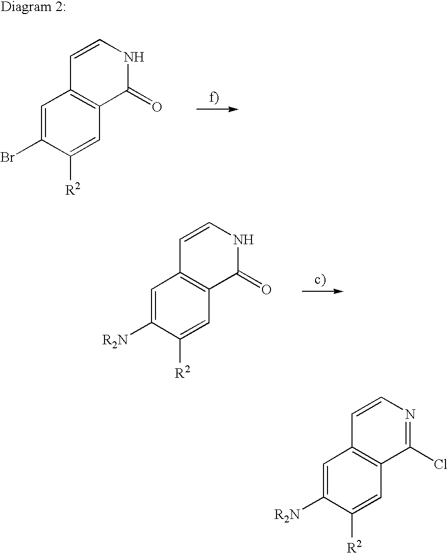
f) (Ph₃P)₄Pd, Cs₂CO₃, CH₃CN; C) SOCl₂ or SOBr₂ or Cl(CO)(CO)Cl

A compound of the above general formula (III), wherein
R¹ and R² are as hereinbefore defined,
X denotes a nitrogen atom
Y and Z each denote a methyne group,
L denotes a leaving group such as a halogen atom, for example a chlorine or bromine atom,
may be prepared for example according to the following Diagram 3: 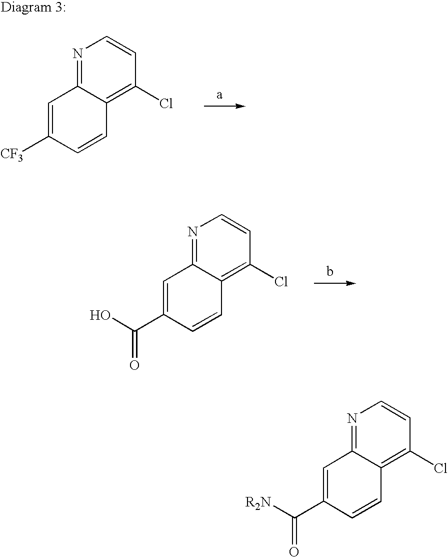
a) conc. H₂SO₄, 550 Watt microwave; b) HNR₂, N-methylmorpholine, propanephosphonic anhydride

Methods of amide coupling are described for example in P. D. Bailey, I. D. Collier, K. M. Morgan in “Comprehensive Functional Group Interconversions”, Vol. 5, page 257ff., Pergamon 1995.

In the reactions described above any reactive group present such as hydroxy, carboxy, amino, alkylamino or imino groups may be protected during the reaction by conventional protecting groups which are cleaved again after the reaction.

For example, a suitable protecting group for a hydroxy group may be the methoxy, benzyloxy, trimethylsilyl, acetyl, benzoyl, tert.butyl, trityl, benzyl or tetrahydropyranyl group,
suitable protecting groups for a carboxyl group might be the trimethylsilyl, methyl, ethyl, tert.butyl, benzyl or tetrahydropyranyl group,
suitable protecting groups for an amino, alkylamino or imino group might be the acetyl, trifluoroacetyl, benzoyl, ethoxycarbonyl, tert.butoxycarbonyl, benzyloxycarbonyl, benzyl, methoxybenzyl or 2,4-dimethoxybenzyl group and additionally, for the amino group, the phthalyl group.

Other protective groups and their cleaving are described in T. W. Greene, P. G. M. Wuts, “Protecting Groups in Synthesis”, Wiley, 1991.

Any protecting group used may optionally subsequently be cleaved for example by hydrolysis in an aqueous solvent, e.g. in water, isopropanol/water, tetrahydrofuran/water or dioxane/water, in the presence of an acid such as trifluoroacetic acid, hydrochloric acid or sulphuric acid or in the presence of an alkali metal base such as lithium hydroxide, sodium hydroxide or potassium hydroxide or by ether splitting, e.g. in the presence of iodotrimethylsilane, at temperatures between 0 and 100° C., preferably at temperatures between 10 and 50° C.

However, a benzyl, methoxybenzyl or benzyloxycarbonyl group is cleaved hydrogenolytically, for example, e.g. with hydrogen in the presence of a catalyst such as palladium/charcoal in a solvent such as methanol, ethanol, ethyl acetate, dimethylformamide, dimethylformamide/acetone or glacial acetic acid, optionally with the addition of an acid such as hydrochloric acid at temperatures between 0 and 50° C., but preferably at ambient temperature, and at a hydrogen pressure of 1 to 7 bar, preferably, however, 1 to 5 bar.

A methoxybenzyl group may also be cleaved in the presence of an oxidising agent such as cerium(IV)ammonium nitrate in a solvent such as methylene chloride, acetonitrile or acetonitrile/water at temperatures of between 0 and 50° C., but preferably at ambient temperature.

A methoxy group is expediently cleaved in the presence of boron tribromide in a solvent such as methylene chloride at temperatures between −35 and −25° C.

A 2,4-dimethoxybenzyl group is preferably cleaved in trifluoroacetic acid in the presence of anisol.

A tert.butyl or tert.butyloxycarbonyl group is preferably cleaved by treating with an acid such as trifluoroacetic acid or hydrochloric acid, optionally using a solvent such as methylene chloride, dioxane or ether.

A phthalyl group is preferably cleaved in the presence of hydrazine or a primary amine such as methylamine, ethylamine or n-butylamine in a solvent such as methanol, ethanol, isopropanol, toluene/water or dioxan at temperatures between 20 and 50° C.

An allyloxycarbonyl group is cleaved by treating with a catalytic amount of tetrakis-(triphenylphosphine)-palladium(0), preferably in a solvent such as tetrahydrofuran and preferably in the presence of an excess of a base such as morpholine or 1,3-dimedone at temperatures between 0 and 100° C., preferably at ambient temperature and under an inert gas, or by treating with a catalytic amount of tris-(triphenylphosphine)-rhodium(l)chloride in a solvent such as aqueous ethanol and optionally in the presence of a base such as 1,4-diazabicyclo[2.2.2]octane at temperatures between 20 and 70° C.

Moreover the compounds of general formula I obtained may be resolved into their enantiomers and/or diastereomers.

Thus, for example, the compounds of general formula I obtained which occur as racemates may be separated by methods known per se (cf. Allinger N. L. and Eliel E. L. in “Topics in Stereochemistry”, Vol. 6, Wiley Interscience, 1971) into their optical antipodes and compounds of general formula I with at least 2 asymmetric carbon atoms may be resolved into their diastereomers on the basis of their physical-chemical differences using methods known per se, e.g. by chromatography and/or fractional crystallisation, and, if these compounds are obtained in racemic form, they may subsequently be resolved into the enantiomers as mentioned above.

The enantiomers are preferably separated by column separation on chiral phases or by recrystallisation from an optically active solvent or by reacting with an optically active substance which forms salts or derivatives such as e.g. esters or amides with the racemic compound, particularly acids and the activated derivatives or alcohols thereof, and separating the diastereomeric mixture of salts or derivatives thus obtained, e.g. on the basis of their differences in solubility, whilst the free antipodes may be released from the pure diastereomeric salts or derivatives by the action of suitable agents. Optically active acids in common use are e.g. the D- and L-forms of tartaric acid or dibenzoyltartaric acid, di-o-tolyltartaric acid, malic acid, mandelic acid, camphorsulphonic acid, glutamic acid, aspartic acid or quinic acid. An optically active alcohol may be for example (+) or (−)-menthol and an optically active acyl group in amides may be a (+)- or (−)-menthyloxycarbonyl, for example.

Furthermore, the compounds of formula I may be converted into the salts thereof, particularly for pharmaceutical use into the physiologically acceptable salts with inorganic or organic acids. Acids which may be used for this purpose include for example hydrochloric acid, hydrobromic acid, sulphuric acid, methanesulphonic acid, phosphoric acid, fumaric acid, succinic acid, lactic acid, citric acid, tartaric acid or maleic acid.

Moreover, if the new compounds of formula I contain a carboxy group, they may subsequently, if desired, be converted into the salts thereof with inorganic or organic bases, particularly for pharmaceutical use into the physiologically acceptable salts thereof. Suitable bases for this purpose include for example sodium hydroxide, potassium hydroxide, cyclohexylamine, ethanolamine, diethanolamine and triethanolamine.

As already mentioned, the new compounds of general formula I as well as the tautomers, the enantiomers, the diastereomers and the physiologically acceptable salts thereof have valuable properties, particularly an antithrombotic activity, which is preferably based on an effect on thrombin or factor Xa, for example on a thrombin-inhibiting or factor Xa-inhibiting activity, on a prolonging effect on the aPTT time and on an inhibiting effect on related serine proteases such as e.g. urokinase, factor VIIa, factor IX, factor XI and factor XII.

The compounds listed as examples were investigated for their effect on the inhibition of factor Xa as follows:

Method:

Enzyme-kinetic measurement with chromogenic substrate. The quantity of p-nitroaniline (pNA) released from the colourless chromogenic substrate by human factor Xa is determined photometrically at 405 nm. It is proportional to the activity of the enzyme used. The inhibition of the enzyme activity by the test substance (in relation to the solvent control) is determined at various concentrations of test substance and from this the IC₅₀ is calculated, as the concentration which inhibits the factor Xa used by 50%.

Material:

Tris(hydroxymethyl)-aminomethane buffer (100 mMol) and sodium chloride (150 mMol), pH 8.0 plus 1 mg/ml Human Albumin Fraction V, protease-free

FactorXa (Calbiochem), spec. activity: 217 IU/mg, final concentration: 7 IU/ml for each reaction mixture

Substrate S 2765 (Chromogenix), final concentration: 0.3 mM/l (1 KM) for each reaction mixture

Test substance: final concentration 100, 30, 10, 3, 1, 0.3, 0.1, 0.03, 0.01, 0.003, 0.001 μMol/l

Procedure:

10 μl of a 23.5-times concentrated starting solution of the test substance or solvent (control), 175 μl of TRIS/HSA buffer and 25 μl of a 65.8 U/L Factor Xa working solution are incubated for 10 minutes at 37° C. After the addition of 25 μl of S 2765 working solution (2.82 mMol/l) the sample is measured in a photometer (SpectraMax 250) at 405 nm for 600 seconds at 37° C.

Evaluation:

1. Determining the maximum increase (deltaOD/minutes) over 21 measuring points.

2. Determining the % inhibition based on the solvent control.

3. Plotting a dosage/activity curve (% inhibition vs substance concentration).

4. Determining the IC₅₀ by interpolating the X-value (substance concentration) of the dosage/activity curve at Y=50% inhibition.

All the compounds tested had an IC₅₀ value of less than 100 μmol/L.

The compounds prepared according to the invention are generally well tolerated.

In view of their pharmacological properties the new compounds and the physiologically acceptable salts thereof are suitable for the prevention and treatment of venous and arterial thrombotic diseases, such as for example the prevention and treatment of deep leg vein thrombosis, for preventing reocclusions after bypass operations or angioplasty (PT(C)A), and occlusion in peripheral arterial diseases, and for preventing and treating pulmonary embolism, disseminated intravascular coagulation, for preventing and treating coronary thrombosis, for preventing stroke and the occlusion of shunts. In addition, the compounds according to the invention are suitable for antithrombotic support in thrombolytic treatment, such as for example with alteplase, reteplase, tenecteplase, staphylokinase or streptokinase, for preventing long-term restenosis after PT(C)A, for the prevention and treatment of ischaemic incidents in patients with all forms of coronary heart disease, for preventing metastasis and the growth of tumours and inflammatory processes, e.g. in the treatment of pulmonary fibrosis, for preventing and treating rheumatoid arthritis, for preventing and treating fibrin-dependent tissue adhesions and/or the formation of scar tissue and for promoting wound healing processes. The new compounds and the physiologically acceptable salts thereof may be used therapeutically in conjunction with acetylsalicylic acid, with inhibitors of platelet aggregation such as fibrinogen receptor antagonists (e.g. abciximab, eptifibatide, tirofiban, roxifiban), with physiological activators and inhibitors of the clotting system and the recombinant analogues thereof (e.g. Protein C, TFPI, antithrombin), with inhibitors of ADP-induced aggregation (e.g. clopidogrel, ticlopidine), with P₂T receptor antagonists (e.g. cangrelor) or with combined thromboxane receptor antagonists/synthetase inhibitors (e.g. terbogrel).

The dosage required to achieve such an effect is appropriately 0.01 to 3 mg/kg, preferably 0.03 to 1.0 mg/kg by intravenous route, and 0.03 to 30 mg/kg, preferably 0.1 to 10 mg/kg by oral route, in each case administered 1 to 4 times a day.

For this purpose, the compounds of formula I prepared according to the invention may be formulated, optionally together with other active substances, with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof, to produce conventional galenic preparations such as plain or coated tablets, capsules, powders, suspensions or suppositories.

The Examples which follow are intended to illustrate the invention without restricting its scope:

The HPLC/MS data were obtained under the following conditions:
(a) Waters ZMD, Alliance 2690 HPLC, Waters 2700 Autosampler, Waters 996 diode array detector

The following was used as the mobile phase:
A: water with 0.1% trifluoroacetic acid
B: acetonitrile with 0.1% trifluoroacetic acid

The following gradient was used:

| time in min | % A | % B | flow rate in ml/min |
|---|---|---|---|
| 0.0 | 95 | 5 | 1.00 |
| 0.1 | 95 | 5 | 1.00 |
| 5.1 | 2 | 98 | 1.00 |
| 6.5 | 2 | 98 | 1.00 |
| 7.0 | 95 | 5 | 1.00 |

The stationary phase used was a Waters column X-Terra™ MS C₁₈ 3.5 μm, 4.6 mm×50 mm (column temperature: constant at 25° C.)

The diode array detection took place in a wavelength range from 210-500 nm

### EXAMPLE 1

### 3-{[7-(2,5-dihydropyrrol-1-yl-carbonyl)-quinazolin-4-yl]aminomethyl}-4-hydroxy-benzamidine-hydrochloride

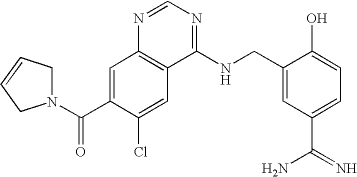

50 mg (97 μmol) 4-benzyloxy-3-{[7-(2,5-dihydro-pyrrol-1-ylcarbonyl)-quinazolin-4-yl]aminomethyl}-benzamidine-hydrochloride [prepared by coupling 4-chloro-7(2,5-dihydropyrrolidin-1-yl-carbonyl)quinazoline (synthesis analogous to 8c) and 3-aminomethyl-4-benyloxy-benzonitrile (=Example 2d) analogously to Example 8h and subsequent Pinner reaction analogously to Example 2h] are dissolved in 5 ml trifluoroacetic acid and 0.6 ml thioanisol and stirred for four days at 50° C. The solvent is distilled off and the residue is dissolved in methanol. When ethereal hydrochloric acid is added a brown solid separates off, which is dissolved twice in methanol and precipitated with diethyl ether.

Yield: 33.5 mg (81% of theory)

R_{f} value: 0.69 (silica gel; acetonitrile/chloroform/water/formic acid 75:20:10:15)

C₂₁H₂₀N₆O₂×HCl (388.43/424.89)

Mass spectrum: (M+H)⁺=389

### EXAMPLE 2

### 4-hydroxy-3-{[7-(pyrrolidin-1-yl-carbonyl)-quinolin-4-yl]aminomethyl}-benzamidine

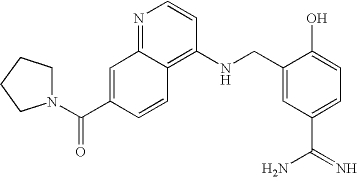

### a. 2-benzyloxy-5-bromo-benzaldehyde

10.2 g (50 mmol) 5-bromo-2-hydroxy-benzaldehyde are dissolved in 230 ml N,N-dimethylformamide and after the addition of 6.9 g (50 mmol) potassium carbonate stirred for 15 minutes. Then 5.9 ml (50 mmol) benzylbromide are added dropwise and the mixture is stirred for 5 hours at ambient temperature. It is then poured onto ice water and extracted with dichloromethane. The combined organic extracts are dried and concentrated by evaporation.

Yield: 12.7 g (87% of theory)

R_{f} value: 0.59 (silica gel; petroleum ether/ethyl acetate=4:1)

C₁₄H₁₁BrO₂ (291.14)

Mass spectrum: (M+H)⁺=291/93 (bromine isotope)

### b. 4-benzyloxy-3-formyl-benzonitrile

5.0 g (17.1 mmol) 2-benzyloxy-5-bromo-benzaldehyde are dissolved in 115 ml of dimethylformamide and after the addition of 3.0 g (34.3 mmol) copper-(I)-cyanide and 491 mg (0.42 mmol) tetrakis-triphenylphosphine-palladium-(0) the mixture is stirred for 24 hours at 145° C. After standing overnight the solvent is distilled off, the residue is suspended in ethyl acetate and suction filtered. The filtrate is washed with 10% ammonia solution, the organic phase is separated off, dried and concentrated by evaporation. The residue is chromatographed on silica gel, eluting with petroleum ether/ethyl acetate (4:1 and 7:3).

Yield: 2.4 g (59% of theory)

R_{f} value: 0.77 (silica gel; petroleum ether/ethyl acetate=19:1)

C₁₅H₁₁NO₂ (237.26)

Mass spectrum: (M+H)⁺=238

### c. 4-benzyloxy-3-tert.-butoxycarbonylaminomethyl-benzonitrile

15.0 g (63.2 mmol) 4-benzyloxy-3-formyl-benzonitrile are dissolved in 300 ml acetonitrile and after the addition of 21.6 g (117 mmol) tert.-butylcarbamate, 29.7 ml (116 mmol) triethylsilane and 9.4 ml (114 mmol) trifluoroacetic acid stirred for 2 hours at ambient temperature. Then diethyl ether is added and the mixture is washed with sodium hydrogen carbonate solution. The combined organic extracts are dried and concentrated by evaporation.

Yield: 40.00 g (quantitative)

R_{f} value: 0.32 (silica gel; petroleum ether/ethyl acetate=4:1+0.5% ammonia solution)

C₂₀H₂₂N₂O₃ (338.41)

Mass spectrum: (M+H)⁺=339

### d. 3-aminomethyl-4-benzyloxy-benzonitrile

5.3 g (15.6 mmol) 4-benzyloxy-3-tert.-butoxycarbonylaminomethyl-benzonitrile are dissolved in 50 ml 1,4-dioxane and after the addition of 35 ml of conc. hydrochloric acid stirred overnight at ambient temperature. Then the mixture is poured onto ice water, made alkaline with ammonia and extracted with ethyl acetate. The combined organic extracts are dried and concentrated by evaporation. The residue is chromatographed on silica gel, eluting with ethyl acetate/1% ammonia.

Yield: 2.0 g (54% of theory)

R_{f} value: 0.10 (silica gel; petroleum ether/ethyl acetate=1:1+1% ammonia solution)

C₁₅H₁₄N₂O (238.29)

Mass spectrum: (M+H)⁺=239

### e. 4-chloro-quinoline-7-carboxylic acid

10.0 g (43.2 mmol) of 4-chloro-7-trifluoromethyl-quinoline are irradiated in 200 ml of conc. sulphuric acid for 2 hours in the microwave at 550 Watt. The reaction solution is poured onto ice water and adjusted to pH 3-4 with sodium hydroxide solution. The precipitated product is suction filtered, washed with water and dried.

Yield: 8.9 g (99% of theory)

R_{f} value: 0.45 (silica gel; toluene/ethanol=4:1)

C₁₀H₆ClNO₂ (207.62)

Mass spectrum: (M+H)⁺=208/10 (chlorine isotope)

### f. 4-chloro-7-(pyrrolidin-1-ylcarbonyl)-quinoline

5.2 g (25 mmol) 4-chloro-quinoline-7-carboxylic acid and 2.1 ml (25 mmol) pyrrolidine are suspended in 125 ml of ethyl acetate, 14.0 ml (128 mmol) N-methylmorpholine is added and then 29.2 ml (50.8 mmol) propanephosphonic anhydride are added dropwise. After 40 hours the mixture is washed with 20 ml sodium hydrogen carbonate solution and extracted with ethyl acetate. The combined organic extracts are dried and concentrated by evaporation. The residue is chromatographed on silica gel, eluting with toluene/ethanol 95:5.

Yield: 4.7 g (72% of theory)

R_{f} value: 0.18 (silica gel; toluene/ethanol=9:1)

C₁₄H₁₃ClN₂O (260.72)

Mass spectrum: (M+H)⁺=261/63 (chlorine isotope)

### g. 4-benzyloxy-3-{[7-(pyrrolidin-1-ylcarbonyl)-quinolin-4-yl]aminomethyl}-benzonitrile

395 mg (1.51 mmol) of 4-chloro-7-(pyrrolidin-1-ylcarbonyl)-quinoline and 360 mg (1.51 mmol) of 3-aminomethyl-4-benzyloxy-benzonitrile are heated to 100° C. for 16 hours. The residue is cooled and chromatographed on silica gel, eluting with toluene/ethanol 4:1.

Yield: 600 mg (86% of theory)

C₂₉H₂₆N₄O₂ (462.55)

Mass spectrum: (M+H)⁺=463

### h. 4-benzyloxy-3-{[7-(pyrrolidin-1-yl-carbonyl)-quinolin-4-yl]aminomethyl}-benzamidine-hydrochloride

200 mg (0.43 mmol) of 4-benzyloxy-3-{[7-(pyrrolidin-1-ylcarbonyl)-quinolin-4-yl]aminomethyl}-benzonitrile are dissolved in 5.5 ml of saturated ethanolic hydrochloric acid and stirred for 24 hours at ambient temperature. The solvent is distilled off, the residue is dissolved in 10 ml absolute ethanol and combined with 418 mg (4.36 mmol) of ammonium carbonate. After 16 hours it is filtered off, washed with ethanol, the filtrate is evaporated to dryness and the residue is purified by chromatography on silica gel (gradient: dichloromethane/ethanol/concentrated ammonia solution 7:3:0.05→methanol/concentrated ammonia solution 10:1).

Yield: 102 mg (46% of theory)

C₂₉H₂₉N₅O₂×HCl (479.58/516.05)

Mass spectrum: (M+H)⁺=480

### i. 4-hydroxy-3-{[7-(pyrrolidin-1-yl-carbonyl)-quinolin-4-yl]aminomethyl}-benzamidine-hydrochloride

100 mg (0.194 mmol) of 4-benzyloxy-3-{[7-(pyrrolidin-1-yl-carbonyl)-quinolin-4-yl]aminomethyl}-benzamidine-hydrochloride are dissolved in 25 ml of methanol and after the addition of 70 mg of palladium on activated charcoal hydrogenated with hydrogen at ambient temperature. Then the catalyst is filtered off and the solution is concentrated by evaporation. The residue is purified by chromatography on silica gel (eluant: dichloromethane/methanol/concentrated ammonia solution 50:50:0.04).

Yield: 53 mg (64% of theory).

R_{f} value: 0.21 (silica gel; dichloromethane/methanol=1:1+0.1% ammonia solution)

C₂₂H₂₄N₅O₂×HCl (389.47/427.92)

Mass spectrum: (M+H)⁺=390
(M−H)⁻=388

### EXAMPLE 3

### 3-{[7-(pyrrolidin-1-yl-carbonyl)-quinolin-4-yl]aminomethyl}-benzamidine

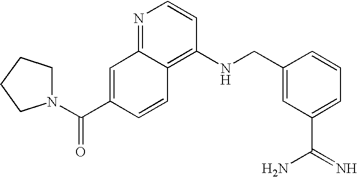

Prepared analogously to Example 2h from 3-{[7-(pyrrolidin-1-yl-carbonyl)-quinolin-4-yl]aminomethyl}-benzonitrile and ethanolic hydrochloric acid/ammonium carbonate in ethanol.

Yield: 34% of theory

R_{f} value: 0.14 (silica gel; dichloromethane/methanol=1:1+0.1% ammonia solution)

C₂₂H₂₃N₅O (373.45)

Mass spectrum: (M+H)⁺=374
(M−H)⁻=372

### EXAMPLE 4

### 3-{2-phenyl-1-[7-(pyrrolidin-1-yl-carbonyl)-quinolin-4-ylamino]-ethyl}-benzamidine

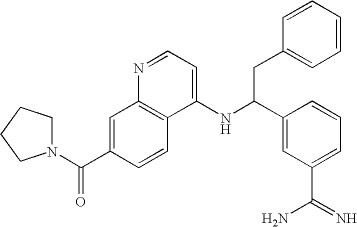

### a. 3-phenylmethylcarbonyl-benzonitrile

10.0 g (60.4 mmol) of 3-cyanobenzoylchloride, 7.7 ml (64.7 mmol) of benzylbromide and 2.0 g (2.85 mmol) of bis-(triphenylphosphine)-palladium-(II)-chloride are placed in 300 ml of tetrahydrofuran and combined with 7.5 g (114.7 mmol) of zinc in batches while cooling with ice. Then the mixture is stirred for 30 minutes while cooling with ice and for 4 hours at ambient temperature. The zinc is suction filtered and the solution is concentrated by evaporation. The residue is chromatographed on silica gel, eluting with toluene/acetone 100:1.

Yield: 6.8 g (41% of theory)

R_{f} value: 0.91 (silica gel; dichloromethane/methanol=99:1)

C₁₅H₁₁NO (221.26)

Mass spectrum: (M−H)⁻=220

### b. 3-(1-amino-2-phenyl-ethyl)-benzonitrile

6.8 g (24.6 mmol) of 3-phenylmethylcarbonyl-benzonitrile are dissolved in 160 ml of methanol and after the addition of 19.3 g (245.9 mmol) of ammonium acetate, 1.6 g (24.6 mmol) of sodium cyanoborohydride and 15.8 g of 4 Å molecular sieve, stirred for 15 minutes at ambient temperature and refluxed for 4 hours. After cooling the mixture is poured onto water and extracted with ethyl acetate. The combined organic extracts are dried and concentrated by evaporation. The residue is chromatographed on silica gel, eluting with dichloromethane/methanol 95:5.

Yield: 2.2 g (40% of theory)

R_{f} value: 0.10 (silica gel; toluene/acetone=19:1) C₁₅H₁₄N₂ (222.29)

Mass spectrum: (M+H)⁺=223

### c. 3-{2-phenyl-1-[7-(pyrrolidin-1-yl-carbonyl)-quinolin-4-yl]amino-ethyl}-benzonitrile

Prepared analogously to Example 2g from 4-chloro-7-(pyrrolidin-1-ylcarbonyl)-quinoline and 3-(1-amino-2-phenyl-ethyl)-benzonitrile at 100° C.

Yield: 85% of theory

R_{f} value: 0.43 (silica gel; toluene/ethanol=4:1)

C₂₉H₂₆N₄O (446.55)

Mass spectrum: (M+H)⁺=447

### d. 3-f2-phenyl-1-[7-(pyrrolidin-1-yl-carbonyl)-quinolin-4-yl]amino-ethyl}-benzamidine

Prepared analogously to Example 2h from 3-{2-phenyl-1-[7-(pyrrolidin-1-yl-carbonyl)-quinolin-4-yl]amino-ethyl}-benzonitrile and ethanolic hydrochloric acid/ammonium carbonate in ethanol.

Yield: 58% of theory

R_{f} value: 0.30 (silica gel; dichloromethane/methanol=1:1+0.1% ammonia solution)

C₂₉H₂₉N₅O (463.58)

Mass spectrum: (M+H)⁺=464

### EXAMPLE 5

### 3-{2-methoxycarbonyl-1-[7-(Pyrrolidin-1-yl-carbonyl)-quinolin-4-ylamino]-ethyl}-benzamidine-hydrochloride

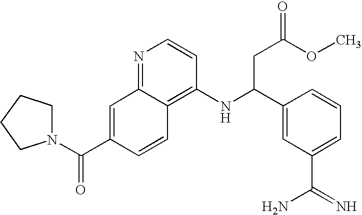

Prepared analogously to Example 2h from 3-{2-methoxycarbonyl-1-[7-(pyrrolidin-1-yl-carbonyl)-quinolin-4-ylamino]-ethyl}-benzonitrile and hydrochloric acid/ammonium carbonate in ethanol.

Yield: 2.5% of theory

R_{f} value: 0.21 (silica gel; dichloromethane/methanol=1:1+0.1% ammonia solution)

C₂₅H₂₇N₅O₃×HCl (445.52/481.98)

Mass spectrum: (M+H)⁺=446
(M−H)⁻=444

### EXAMPLE 6

### 3-{2-hydroxycarbonyl-1-[7-(pyrrolidin -1-yl-carbonyl)-quinolin-4-ylamino]-ethyl}-benzamidine-hydrochloride

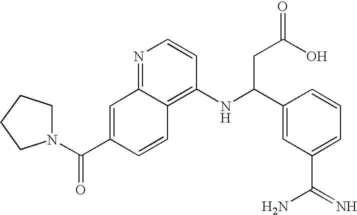

35 mg (0.066 mmol) of 3-{2-methoxycarbonyl-1-[7-(pyrrolidin-1-yl-carbonyl)-quinolin-4-ylamino]-ethyl}-benzamidine-hydrochloride are stirred for 16 hours in 5 ml of 6 molar hydrochloric acid at ambient temperature. Then the solvent is distilled off, combined with toluene and concentrated by evaporation.

Yield: 32 mg (96% of theory)

R_{f} value: 0.2 (silica gel; dichloromethane/methanol/ammonia=8:2:0.01)

C₂₄H₂₅N₅O₃×2 HCl (431.49/504.42)

Mass spectrum: (M+H)⁺=432
(M−H)⁻=430

### EXAMPLE 7

### 4-hydroxy-3-{[7-methoxy-6-(pyrrolidin-1-yl-carbonyl)-isoquinolin-1-yl]aminomethyl}-benzamidine-hydrochloride

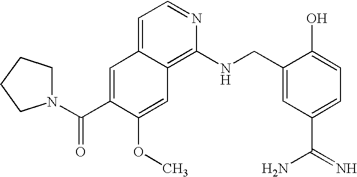

### a. (E)-3-(3-bromo-4-methoxy-phenyl)-acrylic acid

25.0 g (0.116 mol) 3-bromoanisaldehyde are dissolved in 120 ml of pyridine with gentle heating; then 15.6 g (0.150 mol) malonic acid and 5.75 ml (0.058 mol) piperidine are added. Then the mixture is stirred for 2 hours at 100° C. (gas given off) and overnight at ambient temperature. The solvent is distilled off, the residue is combined with 500 ml ice water and adjusted to pH 4-5 with glacial acetic acid. The precipitated product is suction filtered, washed with water and dried.

Yield: 29.7 g (99% of theory)

C₁₀H₉BrO₃ (257.09)

Mass spectrum: (M−H)⁺=255/57 (bromine isotope)

### b. (E)-3-(3-bromo-4-methoxy-phenyl)-acrylic acid azide

14.9 g (58 mmol) of (E)-3-(3-bromo-4-methoxy-phenyl)-acrylic acid are suspended in 285 ml acetone, combined with 8.1 ml (58 mmol) of triethylamine, 8.4 ml (77 mmol) of ethyl chloroformate are added dropwise at −2 to +2° C. and the mixture is stirred for 90 minutes at this temperature. Then a solution of 5.6 g (87 mmol) of sodium azide in 15 ml of water is added dropwise and the mixture is stirred for 1 hour without cooling. The reaction product is stirred into 800 ml of water and suction filtered.

Yield: 15.6 g (86% of theory)

C₁₀H₈BrN₃O₂ (282.09)

Mass spectrum: (M)⁺=281/83 (bromine isotope)

### c. 6-bromo-7-methoxy-2-isoquinolin-1-one

A suspension of 15.5 g (55 mmol) of (E)-3-(3-bromo-4-methoxy-phenyl)-acrylic acid azide in 50 ml diphenylether is added to a solution of 13.1 ml (55 mmol) of tributylamine in 250 ml diphenylether at 235° C. and then the mixture is kept for another 2.5 hours at 235° C. It is then cooled to ambient temperature, stirred into 800 ml n-hexane and the precipitate is suction filtered. The residue is boiled in 400 ml of ethanol, cooled, suction filtered and washed with ethanol/ether and dried.

Yield: 7.5 g (54% of theory)

C₁₀H₈BrNO₂ (254.08)

Mass spectrum: (M+H)⁺=254/56 (bromine isotope)

### d. 6-bromo-1-chloro-7-methoxy-isoquinoline

4.6 g (18 mmol) of 6-bromo-7-methoxy-2-isoquinolin-1-one are stirred in 20 ml phosphorus oxychloride for 1.5 hours at 110° C. The solvent is distilled off, the residue is combined with ice/dichloromethane and adjusted to pH 8 with 10% sodium hydroxide solution. The organic phase is separated off, dried and concentrated by evaporation. The residue is chromatographed on silica gel, eluting with dichloromethane.

Yield: 2.6 g (53% of theory)

C₁₀H₇BrClNO (272.53)

Mass spectrum: (M+H)⁺=272/74/76 (bromine-chlorine isotope)

### e. 1-chloro-7-methoxy-isoquinoline-6-carboxylic Acid

5.8 ml (9.3 mmol) of n-butyllithium (1.6 molar in tetrahydrofuran) are added dropwise to a solution of 2.3 g (8.4 mmol) of 6-bromo-1-chloro-7-methoxy-isoquinoline in 50 ml of tetrahydrofuran at −70° C. under a nitrogen atmosphere. Then dry carbon dioxide is piped in for 40 minutes at −65 to 68° C. The reaction mixture is then allowed to warm up to ambient temperature and combined with ice water. It is then made alkaline with conc. ammonia and extracted with ethyl acetate. The aqueous phase is acidified with conc. hydrochloric acid and extracted with ethyl acetate. The organic extracts are dried and concentrated by evaporation. The residue is chromatographed on silica gel, eluting with 0-5% dichloromethane/(methanol/glacial acetic acid 19:1).

Yield: 950 mg (47% of theory)

### f. 1-chloro-7-methoxy-6-(pyrrolidin-1-yl-carbonyl)-isoquinoline

A solution of 0.94 g (4.0 mmol) of 1-chloro-7-methoxy-isoquinoline-6-carboxylic acid in 5 ml of N,N-dimethylformamide with 0.67 ml (6.0 mmol) of N-methylmorpholine and 0.42 ml (6.0 mmol) of pyrrolidine is combined with 13 g (4.0 mmol) of O-(benzotriazol-1-yl)-N,N,N′,N′-tetramethyluronium tetrafluoroborate under a nitrogen atmosphere, with stirring, at ambient temperature. After 2.5 hours the mixture is poured into ice water, the resulting precipitate is filtered off and washed with a little cold water.

Yield: 1.15 g (35% of theory).

C₁₅H₁₅ClN₂O₂ (290.75)

Mass spectrum: (M+H)⁺=291/93 (chlorine isotope)

### g. 7-methoxy-1-phenoxy-6-(pyrrolidin-1-yl-carbonyl)-isoquinoline

A mixture of 0.34 g (1.17 mmol) of 1-chloro-7-methoxy-6-(pyrrolidin-1-yl-carbonyl)-isoquinoline, 2.2 g (23.4 mmol) of phenol and 0.14 g (2.5 mmol) of powdered potassium hydroxide are stirred for 22 hours at 90° C. under a nitrogen atmosphere. After cooling, ice water is added and the mixture is extracted with ethyl acetate. The organic phase is washed with 1 molar sodium hydroxide solution, dried and concentrated by evaporation. The residue is stirred with ether and suction filtered.

Yield: 0.2 g (49% of theory)

C₂₁H₂₀N₂O₃ (348.405)

Mass spectrum: (M+H)⁺=349

### h. 4-benzyloxy-3-{[7-methoxy-6-(pyrrolidin-1-yl-carbonyl)-isoquinolin-1-yl]amino-methyl}-benzonitrile

170 mg (0.49 mmol) of 7-methoxy-1-phenoxy-6-(pyrrolidin-1-yl-carbonyl)-isoquinoline and 607 mg (2.5 mmol) of 3-aminomethyl-4-benzyloxy-benzonitrile are heated to 168° C. for 8 hours. After cooling the residue is chromatographed on silica gel, eluting with dichloromethane/methanol/ammonia 19:1:0.1 -4:1:0.1.

Yield: 120 mg (50% of theory)

C₃₀H₂₈N₄O₃ (492.58)

Mass spectrum: (M+H)⁺=493

### i. 4-hydroxy-3-{[7-methoxy-6-(pyrrolidin-1-yl-carbonyl)-isoquinolin-1-yl]aminomethyl}-benzamidine-hydrochloride

Prepared analogously to Example 2h from 4-benzyloxy-3-{[7-methoxy-6-(pyrrolidin-1-yl-carbonyl)-isoquinol in-1-yl]amino-methyl}-benzonitrile and hydrochloric acid/ammonium carbonate in ethanol and subsequently reacting analogously to Example 2i with palladium on activated charcoal and hydrogen in methanol.

Yield: 52% of theory

R_{f} value: 0.63 (silica gel; dichloromethane/methanol=3:1+1% acetic acid)

C₂₃H₂₅N₅O₃×HCl (419.48/455.94)

Mass spectrum: (M+H)⁺=420
(M+Cl)⁻=454/56 (chlorine isotope)

### EXAMPLE 8

### 4-hydroxy-3-{2-phenyl-1-[7-(pyrrolidin-1-yl-carbonyl)-quinazolin-4-ylamino]-ethyl}-benzamidine-hydrochloride

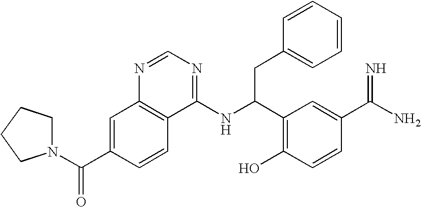

### a. 4-oxo-3,4-dihydro-quinazoline-7-carboxylic Acid

25.0 g (0.13 mol) aminoterephthalic acid are added to 55 ml (1.3 mol) formamide and then stirred for 4.5 hours at 155° C. The reaction mixture is cooled and stirred into ice water. The precipitate is suction filtered and dried at 70° C. in the drying cupboard.

Yield: 22.6 g (86% of theory)

C₉H₆N₂O₃ (190.16)

Mass spectrum: (M+H)⁺=191
(M−H)⁻=189

### b. 4-chloro-quinazoline-7-carboxylic Acid Chloride

1.10 g (5.8 mmol) of 4-oxo-3,4-dihydro-quinazoline-7-carboxylic acid are refluxed for 4 hours in 15 ml of thionyl chloride and 1 ml of dimethylformamide. Then the insoluble matter is filtered off and the filtrate is concentrated by evaporation in vacuo.

Yield: 1.3 g (quantitative)

C₉H₄Cl₂N₂O (227.05)

Mass spectrum: (M)⁺=226/28 (chlorine isotope)

### c. 4-chloro-7-(pyrrolidin-1-yl-carbonyl)-quinazoline

0.37 ml (4.4 mmol) of pyrrolidine are dissolved in 10 ml dichloromethane, a suspension of 1.0 g (4.4 mmol) of 4-chloro-quinazoline-7-carboxylic acid chloride in 35 ml dichloromethane is added dropwise at −50° C. and the mixture is stirred for 5 minutes. Then at −65° C. 0.53 ml (5.2 mmol) of 10 molar sodium hydroxide solution are added dropwise and the mixture is then stirred for another 2 hours without cooling. The solvent is distilled off and the residue is chromatographed on silica gel, eluting with petroleum ether/ethyl acetate=1:1.

R_{f} value: 0.35 (silica gel; ethyl acetate+1% ammonia)

Yield: 0.44 g (38% of theory)

### d. methyl 2-(2-benzyloxy-5-cyano-phenyl)-3-phenyl-propionate

4.6 g (16.3 mmol) of 4-benzyloxy-3-methoxycarbonylmethyl-benzonitrile are dissolved in 10 ml dimethylsulphoxide, combined with 1.9 g (17 mmol) of potassium tert. butoxide and then 2.0 ml (16.8 mmol) of benzylbromide are added dropwise. After 2 hours the mixture is stirred with ice water and extracted with ethyl acetate. The combined organic extracts are dried and concentrated by evaporation. The residue is triturated with diisopropylether and suction filtered.

Yield: 3.7 g (61% of theory)

R_{f} value: 0.43 (silica gel; petroleum ether/ethyl acetate=4:1)

### e. 2-(2-benzyloxy-5-cyano-phenyl)-3-phenyl-propionic Acid

3.7 g (9.9 mmol) of methyl 2-(2-benzyloxy-5-cyano-phenyl)-3-phenyl-propionate are suspended in 40 ml of methanol and after the addition of 20 ml 1 molar sodium hydroxide solution stirred for 1.5 hours at 60° C. Then the methanol is distilled off, the residue is taken up in water and extracted with ether. The aqueous phase is adjusted to pH 6 with glacial acetic acid and the precipitated product is suction filtered.

Yield: 3.3 g (93% of theory)

melting point: 169-171° C.

R_{f} value: 0.2 (Reversed phase RP8; 5% sodium chloride solution/methanol=1:3)

### f. 1-(2-benzyloxy-5-cyano-phenyl)-N-tert.-butoxycarbonyl-2-phenyl-ethylamine

1.4 ml (9.8 mmol) of triethylamine and 2.2 ml (9.8 mmol) of phosphoric acid di-phenylesterazide are added to a suspension of 3.5 g (9.7 mmol) of 2-(2-benzyloxy-5-cyano-phenyl)-3-phenyl-propionic acid in 35 ml tert. butanol at ambient temperature under a nitrogen atmosphere and refluxed for 2.5 hours. Then the mixture is cooled to ambient temperature, combined batchwise with 0.88 g (7.8 mmol) of potassium tert. butoxide and stirred for one hour. It is then poured onto ice water, the precipitate is suction filtered and chromatographed on silica gel, eluting with dichloromethane/methanol+1% ammonia (0-5%).

Yield: 2.2 g (52% of theory)

R_{f} value: 0.3 (silica gel; dichloromethane)

### g. 1-(2-benzyloxy-5-cyano-phenyl)-2-phenyl-ethylamine

Prepared analogously to Example 2d from 1-(2-benzyloxy-5-cyano-phenyl)-N-tert.-butoxycarbonyl-2-phenyl-ethylamine and hydrochloric acid in dioxane.

Yield: 77% of theory

R_{f} value: 0.5 (silica gel; petroleum ether/ethyl acetate/ammonia=1:1:0.1)

### h. 4-benzyloxy-3-{2-phenyl-l-[7-(pyrrolidin-1-yl-carbonyl)-quinazolin-4-ylamino]-ethyl}-benzonitrile

A solution of 350 mg (1.3 mmol) of 4-chloro-7-(pyrrolidin-1-yl-carbonyl)-quinazoline in 5 ml acetonitrile is added dropwise to a solution of 439 mg (1.3 mmol) of 1-(2-benzyloxy-5-cyano-phenyl)-2-phenyl-ethylamine in 5 ml acetonitrile at ambient temperature and after the addition of 0.35 ml (2 mmol) of N,N-diisopropyl-ethylamine the mixture is stirred for 8 hours at 75° C. The solvent is distilled off and the residue is chromatographed on silica gel, eluting with petroleum ether/ethyl acetate +1% ammonia (1:1 and 0:1).

Yield: 0.46 g (62% of theory)

R_{f} value: 0.25 (silica gel; ethyl acetate+1% ammonia)

### i. 4-hydroxy-3-{2-phenyl-1-[7-(Pyrrolidin-1-yl-carbonyl)-quinazolin-4-ylamino]-ethyl}-benzamidine-hydrochloride

Prepared analogously to Example 2h from 4-benzyloxy-3-{2-phenyl-1-[7-(pyrrolidin-1-yl-carbonyl)-quinazolin-4-ylamino]-ethyl}-benzonitrile and hydrochloric acid/ammonium carbonate in ethanol and subsequently reacting analogously to Example 2i with palladium on activated charcoal and hydrogen in methanol.

Yield: 78% of theory (over 2 steps)

R_{f} value: 0.65 (Reversed phase RP8; 5% sodium chloride solution/methanol=1:3)

C₂₈H₂₈N₆O₂×HCl (480.56/517.03)

Mass spectrum: (M+H)⁺=481

### EXAMPLE 9

### 4-hydroxy-3-f[6-methyl-7-(pyrrolidin-1-yl-carbonyl)-quinazolin-4-yl]aminomethyl}-benzamidine-hydrochloride

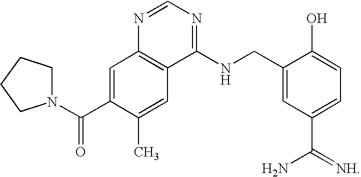

Prepared analogously to Example 2i from 4-benzyloxy-3-{[6-methyl-7-(pyrrolidin-1-yl-carbonyl)-quinazolin-4-yl]aminomethyl}-benzamidine-hydrochloride, palladium on activated charcoal and hydrogen in methanol.

Yield: 87% of theory

R_{f} value: 0.45 (Reversed phase RP8; 5% sodium chloride solution/methanol=1:2)

C₂₂H₂₄N₆O₂×HCl (404.47/440.93)

Mass spectrum: (M+H)⁺=405
(M−H)⁻=403

### EXAMPLE 10

### 4-hydroxy-3-{[7-(pyrrolidin-1-yl-carbonyl)-quinazolin-4-yl]aminomethyl}-benzamidine-hydrochloride

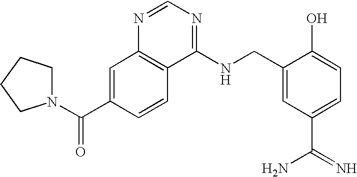

Prepared analogously to Example 2i from 4-benzyloxy-3-{[7-(pyrrolidin-1-yl-carbonyl)-quinazolin-4-yl]aminomethyl}-benzamidine-hydrochloride, palladium on activated charcoal and hydrogen in methanol.

Yield: 83% of theory

R_{f} value: 0.73 (Reversed phase RP8; 5% sodium chloride solution/methanol=1:3)

C₂₁H₂₂N₆O₂×HCl (390.44/426.90)

Mass spectrum: (M+H)⁺=391
(M−H)⁻=389

### EXAMPLE 11

### Ethyl 3-(3-amidino-phenyl)-3-{[6-chloro-7-(pyrrolidin-1-yl-carbonyl)-quinazolin-4-yl]amino}-propionate-acetate

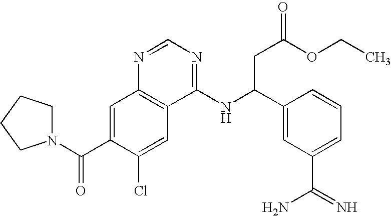

### a. 6-chloro-quinazolin-4-one-7-carboxylic acid-hydrochloride

16.0 g (74.2 mmol) of 2-amino-5-chloro-terephthalic acid are added to 40 ml (1.0 mol) formamide and the mixture is heated to 155° C. for 6 hours with stirring. The mixture is then concentrated in vacuo and at 40° C. combined with 100 ml isopropanol. After 30 minutes the precipitated crystal slurry is filtered off, washed with a 1:1 mixture of ethyl acetate/diethyl ether and dried. 3.25 g of the 11.85 g of solid obtained are then dissolved in 250 ml distilled water and then combined with 25 ml of 2N hydrochloric acid solution. The mixture is diluted with another 200 ml of distilled water and stirred for 30 minutes at ambient temperature. Then the precipitate is filtered off, washed with a little distilled water and dried at 40° C.

Yield: 3.78 g (48%)

R_{f} value: 0.10 (silica gel; dichloromethane/ethanol/glacial acetic acid=4:1:0.1)

C₉H₅CIN₂O₃×HCl (224.60/261.07)

Mass spectrum: (M+H)⁺=225/227 (chlorine isotope)

### b. 4,6-dichloro-7-(pyrrolidin-1-yl-carbonyl)-quinazoline

Prepared by means of a synthesis sequence analogously to Example 8b from 6-chloro-quinazolin-4-one, thionyl chloride and N,N-dimethylformamide and analogously to Example 8c from 4,6-dichloro-quinazoline-7-carboxylic acid chloride, pyrrolidine and 10N sodium hydroxide solution in dichloromethane.

Yield: 37% over 2 steps

R_{f} value: 0.45 (silica gel; dichloromethane)

C₁₃H₁₁Cl₂N₃O (296.16)

Mass spectrum: (M+H)⁺=296/298/300 (chlorine isotope)

### c. ethyl 3-{[6-chloro-7-(pyrrolidin-1-yl-carbonyl)-quinazolin-4-yl]amino}-3-(3-cyano-phenyl)-propionate

592 mg (2.00 mmol) of 4,6-dichloro-7-(pyrrolidin-1-yl-carbonyl)-quinazoline are dissolved together with 459 mg (2.00 mmol) of ethyl 3-amino-3-(3-cyano-phenyl)-propionate in 3 ml of N,N-dimethylformamide under a nitrogen atmosphere and 0.61 ml (2.20 mmol) of triethylamine. The mixture is stirred for 3 hours at ambient temperature. Then it is poured into in ice water, the crystalline precipitate is filtered off, washed with a little distilled water and dried at 40° C. The solid is treated with petroleum ether/diethyl ether 1:1 and dried.

Yield: 430 mg (45% of theory)

R_{f} value: 0.60 (silica gel; dichloromethane/ethanol 9:1)

C₂₅H₂₄CIN₅O₃ (477.96)

Mass spectrum: (M−H)⁺=476/478 (chlorine isotope)

### d. ethyl 3-(3-amidino-phenyl)-3-{[6-chloro-7-(pyrrolidin-1-yl-carbonyl)-quinazolin-4-yl]amino}-propionate-acetate

Prepared analogously to Example 2h from ethyl 3-{[6-chloro-7-(pyrrolidin-1-yl-carbonyl)-quinazolin-4-yl]amino}-3-(3-cyano-phenyl)-propionate and hydrochloric acid/ammonium carbonate in ethanol.

Yield: 25% of theory

R_{f} value: 0.35 (silica gel; dichloromethane/ethanol/glacial acetic acid=4:1:0.1)

C₂₅H₂₇CIN₆O₃×C₂H₄O₂ (494.98/555.03)

Mass spectrum: (M+H)⁺=495/97 (chlorine isotope)
(M−H)⁻=493/95 (chlorine isotope)

### EXAMPLE 12

### 3-(3-amidino-phenyl)-3-{[6-chloro-7-(pyrrolidin-1-yl-carbonyl)-quinazolin-4-yl]amino}-propionic acid-hydrochloride

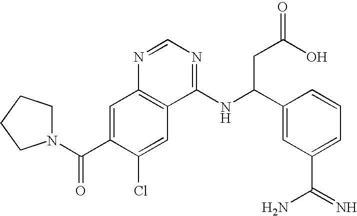

Prepared analogously to Example 6 from ethyl 3-(3-amidino-phenyl)-3-[6-chloro-7-(pyrrolidin-1-yl-carbonyl)-quinazolin-4-yl]amino-propionate-acetate and 6 molar hydrochloric acid.

Yield: 74% of theory

R_{f} value: 0.41 (Reversed phase RP8; 5% sodium chloride solution/methanol=3:2)

C₂₃H₂₃ClN₆O₃×HCl (466.92/503.39)

Mass spectrum: (M+H)⁺=467/69 (chlorine isotope)
(M−H)⁻=465/67 (chlorine isotope)

### EXAMPLE 13

### 3-(3-amidino-phenyl)-3-{[7-(pyrrolidin-1-yl-carbonyl)-quinazolin-4-yl]amino}-propionic acid-hydrochloride

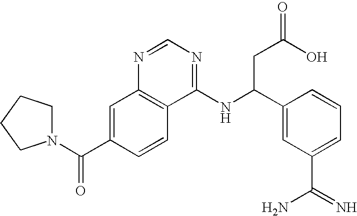

130 mg (0.25 mmol) of ethyl 3-(3-amidino-phenyl)-3-{[7-(pyrrolidin-1-yl-carbonyl)-quinazolin-4-yl]amino}-propionate are suspended in 2 ml of water and stirred with 0.65 ml 1N sodium hydroxide solution for 4.5 h. Then the mixture is adjusted to pH 3 with 1 N HCl, the solvent is distilled off, combined with methanol and filtered. Diethyl ether is added to the filtrate, the precipitate formed is suction filtered and dried in the drying pistol.

Yield: 77% of theory

R_{f} value: 0.5 (Reversed phase RP8; 5% sodium chloride solution/methanol=2:3)

C₂₃H₂₄N₆O₃×HCl (432.48/468.95)

Mass spectrum: (M+H)⁺=433

### EXAMPLE 14

### Ethyl 3-(3-amidino-phenyl)-3-{[7-(thiazolidin-3-yl-carbonyl)-quinazolin-4-yl]amino}-propionate-hydrochloride

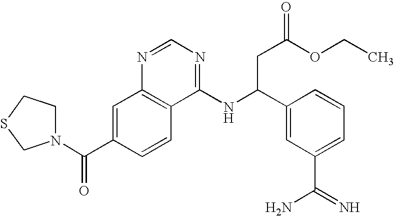

Prepared analogously to Example 2h from ethyl 3-(3-cyano-phenyl)-3-{[7-(thiazolidin-3-yl-carbonyl)-quinazolin-4-yl]amino}-propionate and hydrochloric acid/ammonium carbonate in ethanol.

Yield: 29% of theory

R_{f} value: 0.17 (silica gel; dichloromethane/ethanol=4:1)

C₂₄H₂₆N₆O₃S×HCl (478.57/551.49)

Mass spectrum: (M+H)⁺=479
(M−H)⁻=477

### EXAMPLE 15

### 3-(3-amidino-phenyl)-3-{[7-(thiazolidin-3-yl-carbonyl)-quinazolin-4-yl]amino}-propionic acid-hydrochloride

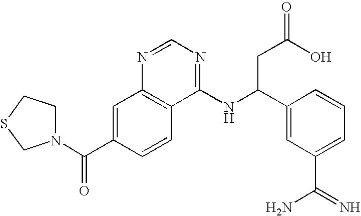

Prepared analogously to Example 6 from ethyl 3-(3-amidino-phenyl)-3-{[7-(thiazolidin-3-yl-carbonyl)-quinazolin-4-yl]amino}-propionate and 6 molar hydrochloric acid.

Yield: 66% of theory

R_{f} value: 0.53 (Reversed phase RP8; 5% sodium chloride solution/methanol 2:3)

C₂₂H22N₆O₃S×HCl (450.52/486.98)

Mass spectrum: (M+H)⁺=451
(M−H)⁻=449

### EXAMPLE 16

### Ethyl 3-(3-amidino-phenyl)-3-{[7-(pyrrolidin-1-yl-carbonyl)-quinazolin-4-yl]amino}-propionate-acetate

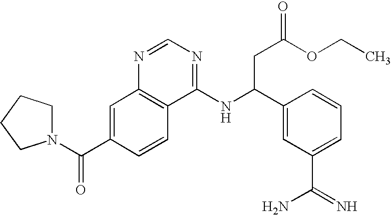

Prepared analogously to Example 2h from ethyl 3-(3-cyano-phenyl)-3-[7-(pyrrolidin-1-yl-carbonyl)-quinazolin-4-ylamino]-propionate and hydrochloric acid/ammonium carbonate in ethanol and subsequent chromatographic purification on silica gel, eluting with methylene chloride: (methanol/acetic acid 19/1) 9:1.

Yield: 76% of theory

R_{f} value: 0.43 (Reversed phase RP8; 5% sodium chloride solution/methanol 2:3)

C₂₅H₂₈N₆O₃×C₂H₄O₂ (460.53/520.59)

Mass spectrum: (M+H)⁺=461

### EXAMPLES 17 and 18

### Rac-3-{[7-(3-benzyloxy-pyrrolidin-1-yl-carbonyl)-quinazolin-4-yl]aminomethyl}-4-hydroxy-benzamidine-acetate (17)

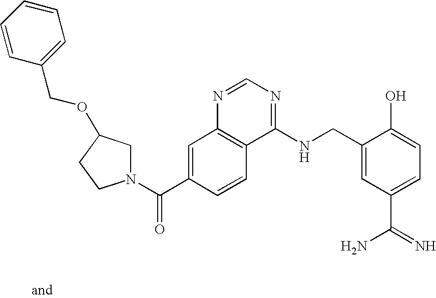

### Rac-3-{[7-(3-hydroxy-pyrrolidin -1-yl-carbonyl)-quinazolin-4-yl]aminomethyl}-4-hydroxy-benzamidine-acetate

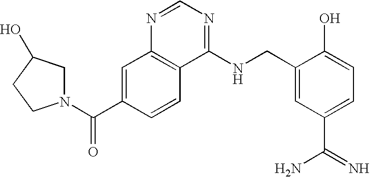

Prepared analogously to Example 2i from 3-{[7-(3-benzyloxy-pyrrolidin-1-yl-carbonyl)-quinazolin-4-yl]aminomethyl}-4-benzyloxy-benzamidine-dihydrochloride, palladium on activated charcoal and hydrogen in methanol and subsequent purification by chromatography on silica gel, eluting with methylene chloride: (methanol/acetic acid 19/1) 9:1→6:1.

Yield 17: 51% of theory

R_{f} value 17: 0.51 (Reversed phase RP8; 5% sodium chloride solution/methanol=1:3)

17: C₂₈H₂₈N₆O₃×C₂H₄O₂ (496.57/557.61)

Mass spectrum: (M+H)⁺=497
(M−H)⁻=495

Yield 18: 9% of theory

R_{f} value 18: 0.66 (Reversed phase RP8; 5% sodium chloride solution/methanol=1:3)

18: C₂₁H₂₂N₆O₃ (406.45)

Mass spectrum: (M+H)⁺=407
(M−H)⁻=405

### EXAMPLE 19

### 3-{[6-(N-acetyl-N-cyclopentylamino)-7-methyl-isoquinolin-1-yl]aminomethyl}-4-hydroxy-benzamidine-hydrochloride

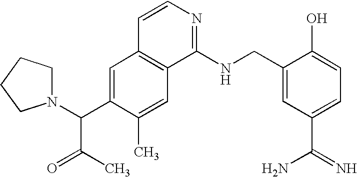

### a. (E)-3-(3-acetylamino-4-methyl-phenyl)-acrylic acid azide

Prepared analogously to Example 7b from (E)-3-(3-acetyl-amino-4-methyl-phenyl)-acrylic acid, ethyl chloroformate, sodium azide and triethylamine in acetone/water.

Yield: 80% of theory

C₁₂H₁₂N₄O₂ (244.25)

Mass spectrum: (M+H)⁺=245
(M−H)⁻=243

### b. N-(7-methyl-1-oxo-1,2-dihydro-isoquinolin-6-yl)-acetamide

Prepared analogously to Example 7c from (E)-3-(3-acetylamino-4-methyl-phenyl)-acrylic acid azide and tributylamine in diphenylether.

Yield: 26% of theory

C₁₂H₁₂N₂O₂ (216.24)

Mass spectrum: (M+H)⁺=217
(M−H)⁻=215

### c. 6-amino-7-methyl-2H-isoquinolin-1-one

3.5 g (16.2 mmol) of N-(7-methyl-1-oxo-1,2-dihydro-isoquinolin-6-yl)-acetamide are suspended in 20 ml of ethanol and after the addition of 4.8 ml conc. hydrochloric acid refluxed for 1 hour. The ethanol is distilled off, the residue dissolved in water, adjusted to pH 10 with conc. ammonia and the precipitate formed is suction filtered.

Yield: 2.4 g (85% of theory)

C₁₀H₁₀N₂O (174.20)

Mass spectrum: (M+H)⁺=175

### d. 6-cyclopentylamino-7-methyl-2H-isoquinolin-1-one

2.4 ml (27.6 mmol) of cyclopentanone and 2.4 g (13.8 mmol) of 6-amino-7-methyl-2H-isoquinolin-1-one are suspended in 25 ml of tetrahydrofuran and after the addition of 0.95 ml (16.6 mmol) of glacial acetic acid and 7.0 g (33.2 mmol) of sodium triacetoxyborohydride refluxed for 4 hours. After standing overnight the solvent is distilled off, the residue is combined with water and extracted with ethyl acetate. The combined organic phases are washed with sodium chloride, dried and concentrated by evaporation. The crude product is chromatographed on silica gel, eluting with petroleum ether/ethyl acetate (3:2 and 0:1).

Yield: 1.2 g (36% of theory)

C₁₅H₁₈N₂O (242.32)

Mass spectrum: (M+Na)⁺=265

### e. N-(1-chloro-7-methyl-isoquinolin-6-yl)-cyclopentylamine

Prepared analogously to Example 7d from 6-cyclopentylamino-7-methyl-2H-isoquinolin-1-one and phosphorus oxychloride.

Yield: 85% of theory

C₁₅H₁₇ClN₂ (260.77)

Mass spectrum: (M+H)⁺=261/63 (chlorine isotope)

### f. N-(1-chloro-7-methyl-isoquinolin-6-yl)-N-cyclopentyl-acetamide

0.95 g (3.6 mmol) of N-(1-chloro-7-methyl-isoquinolin-6-yl)-cyclopentylamine are dissolved in 10 ml of tetrahydrofuran and after the addition of 3.2 g (65.6 mmol) of sodium hydride (50% solution in oil) stirred for 30 minutes at 40° C. Then 7 ml (98.3 mmol) of acetylchloride are added and the mixture is stirred for 2.5 hours at 70° C. Then the solvent is distilled off, the residue is combined with water and extracted with ethyl acetate. The combined organic extracts are dried and concentrated by evaporation. The crude product is chromatographed on silica gel, eluting with petroleum ether/ethyl acetate (7:3 and 3:2).

Yield: 0.8 g (72% of theory)

C₁₇H₁₉ClN₂O (302.91)

Mass spectrum: (M+H)⁺=303/05 (chlorine isotope)

### g. N-cyclopentyl-N-(7-methyl-1-phenoxy-isoquinolin-6-yl)-acetamide

Prepared analogously to Example 7g from N-(1-chloro-7-methyl-isoquinolin-6-yl)-N-cyclopentyl-acetamide and potassium hydroxide in phenol.

Yield: 99% of theory

C₂₃H₂₄N₂O₂ (360.46)

Mass spectrum: (M+H)⁺=361

### h. 3-{[6-(N-acetyl-cyclopentylamino)-7-methyl-isoquinolin-1-yl]aminomethyl}-4-benzyloxy-benzonitrile

Prepared analogously to Example 7h from N-cyclopentyl-N-(7-methyl-1-phenoxy-isoquinolin-6-yl)-acetamide and 3-aminomethyl-4-benzyloxy-benzonitrile.

Yield: 59% of theory

C₃₂H₃₂N₄O₂ (504.63)

Mass spectrum: (M+H)⁺=505
(M−H)⁻=503

### i. 3-{[6-(N-acetyl-cyclopentyl-amino)-7-methyl-isoquinolin-1-yl]aminomethyl}-4-hydroxy-benzamidine-hydrochloride

Prepared analogously to Example 2h from 4-benzyloxy-3-{[6-(N-acetyl-cyclopentylamino)-7-methyl-isoquinolin-1-yl]aminomethyl}-benzonitrile and hydrochloric acid/ammonium carbonate in ethanol and subsequently reacting analogously to Example 2i with palladium on activated charcoal/hydrogen in methanol.

Yield: 77% of theory over 2 steps

R_{f} value: 0.47 (Reversed phase RP8; 5% sodium chloride solution/methanol=1:3)

C₂₅H₂₉N₅O₂×HCl (431.54/468.00)

Mass spectrum: (M+H)⁺=432
(M−H)⁻=430

### EXAMPLE 20

### N-benzoyl-4-hydroxy-3-{[7-(pyrrolidin-1-yl-carbonyl)-quinazolin-4-yl]aminomethyl}-benzamidine

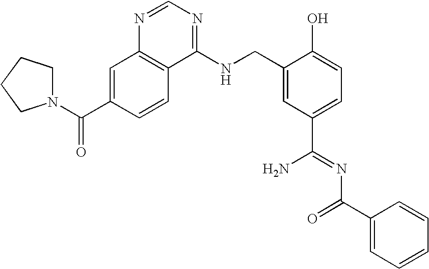

853 mg (2.00 mmol) of 4-hydroxy-3-{[7-(pyrrolidin-1-yl-carbonyl)-quinazolin-4-yl]aminomethyl}-benzamidine-hydrochloride are suspended in 2.5 ml of N,N-dimethylformamide, combined with 0.84 ml (6.0 mmol) of triethylamine and stirred for 5 minutes. Then a solution of 486 mg (2.00 mmol) of 4-nitrophenylbenzoic acid in 7.5 ml N,N-dimethylformamide is added dropwise at 10° C. and the mixture is stirred for 30 minutes at ambient temperature. It is then poured onto ice water and extracted with dichloromethane. The combined organic extracts are dried and concentrated by evaporation. The residue is chromatographed on silica gel, eluting with ethyl acetate/ethanol 9:1 plus 7.5% ammonia (5-10%).

Yield: 70 mg (7% of theory)

R_{f} value: 0.45 (silica gel; ethyl acetate/ethanol=9:1+a few drops of ammonia)

C₂₈H₂₆N₆O₃ (494.55)

Mass spectrum: (M+H)⁺=495
(M−H)⁻=493

### EXAMPLE 21

### N,N′-Dibenzoyl-4-hydroxy-3-{[7-(pyrrolidin-1-yl-carbonyl)-quinazolin-4-yl]aminomethyl}-benzamidine-hydrochloride

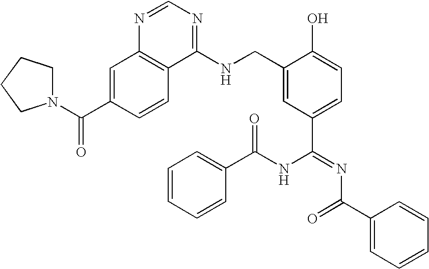

426 mg (1.00 mmol) of 4-hydroxy-3-{[7-(pyrrolidin-1-yl-carbonyl)-quinazolin-4-yl]aminomethyl}-benzamidine-hydrochloride are suspended in 10 ml of water and 15 ml acetone, combined with 0.38 ml (3.3 mmol) of benzoylchloride and stirred for 1 hour at ambient temperature. Then 0.54 g (4.0 mmol) of potassium carbonate are added and the mixture is stirred for 16 hours. The acetone is distilled off, the residue is triturated with water and suction filtered. The crude product is chromatographed on silica gel, eluting with ethyl acetate/ethanol 9:1 plus 7.5% ammonia (2-20%).

Yield: 90 mg (15% of theory)

R_{f} value: 0.59 (silica gel; ethyl acetate/ethanol=9:1+a few drops of ammonia)

C₃₅H₃₀N₆O₄ (598.66)

Mass spectrum: (M+H)⁺=599

### EXAMPLE 22

### N-hydroxy-4-hydroxy-3-{[6-methyl-7-(pyrrolidin-1-yl-carbonyl)-quinazolin-4-yl]aminomethyl}-benzamidine

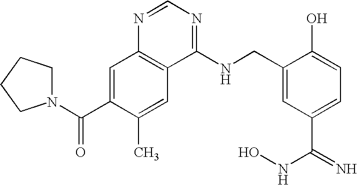

### a. N-hydroxy-4-benzyloxy-3-{[6-methyl-7-(pyrrolidin-1-yl-carbonyl)-quinazolin-4-yl]aminomethyl}-benzamidine

0.300 g (0.628 mmol) of 4-benzyloxy-3-{[6-methyl-7-(pyrrolidin-1-yl-carbonyl)-quinazolin-4-yl]aminomethyl}-benzonitrile are dissolved in 15 ml of methanol and combined successively with 0.125 g (1.52 mmol) of sodium acetate in 0.25 ml of water and 0.106 g (1.52 mmol) of hydroxylamine hydrochloride in 0.25 ml of water and heated to boiling. After 2 hours another 0.2 g sodium acetate and 0.18 g hydroxylamine hydrochloride are added and the mixture is heated to boiling for a further 3 hours. The solvent is distilled off and the residue purified by chromatographing twice on silica gel (1st column: methylene chloride: (methanol/ammonia 19/1) 100:0→70:30; 2nd column: methylene chloride/methanol 98:2→94:6).

Yield: 50 mg (16% of theory)

R_{f} value: 0.55 (silica gel; methylene chloride/methanol=9:1)

C₂₉H₃₀N₆O₃ (510.60)

Mass spectrum: (M+H)⁺=511
(M−H)⁻=509
(M+HCOO)⁻=555

### b. N-hydroxy-4-hydroxy-3-{[6-methyl-7-(pyrrolidin-1-yl-carbonyl)-quinazolin-4-yl]aminomethyl}-benzamidine

Prepared analogously to Example 2i from N-hydroxy-4-benzyloxy-3-{[6-methyl-7-(pyrrolidin-1-yl-carbonyl)-quinazolin-4-yl]aminomethyl}-benzamidine, palladium on activated charcoal and hydrogen in methanol.

Yield: 97% of theory

R_{f} value: 0.45 (silica gel; methylene chloride/methanol=9:1)

C₂₂H₂₄N₆O₃ (420.48)

Mass spectrum: (M+H)⁺=421

(M−H)⁻=419

### EXAMPLE 23

### N-acetoxymethoxycarbonyl-4-hydroxy-3-{[6-methyl-7-(pyrrolidin-1-yl-carbonyl)-quinazolin-4-yl]aminomethyl}-benzamidine

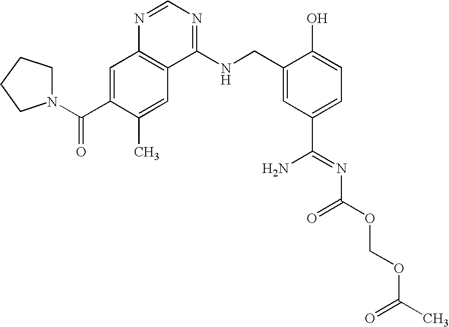

### a. N-acetoxymethoxycarbonyl-4-benzyloxy-3-{[6-methyl-7-(pyrrolidin-1-yl-carbonyl)-quinazolin-4-yl]aminomethyl}-benzamidine

0.495 g (1.00 mmol) of 4-benzyloxy-3-{[6-methyl-7-(pyrrolidin-1-yl-carbonyl)-quinazolin-4-yl]aminomethyl}-benzamidine are suspended in 25 ml methylene chloride, combined with 0.42 ml (3.0 mmol) of triethylamine and 0.306 g (1.20 mmol) of 4-acetoxymethoxycarbonyloxy-nitrobenzene and heated to boiling for 2.5 hours. The reaction mixture is concentrated down to about 3 ml and purified by chromatography with silica gel (methylene chloride/methanol 19:1).

Yield: 300 mg (49% of theory)

R_{f} value: 0.69 (silica gel; ethyl acetate/ethanol=9:1+a few drops of ammonia)

C₃₃H34N₆O₆ (610.66)

Mass spectrum: (M+H)⁺=611
(M+HCOO)⁻=655

### b. N-acetoxymethoxycarbonyl-4-hydroxy-3-{[6-methyl-7-(pyrrolidin-1-carbonyl)-quinazolin4-yl]aminomethyl}-benzamidine

Prepared analogously to Example 2i from N-acetoxymethoxycarbonyl-4-benzyloxy-3-{[6-methyl-7-(pyrrolidin-1-yl-carbonyl)-quinazolin-4-yl]aminomethyl}-benzamidine, palladium on activated charcoal and hydrogen in methanol.

Yield: 30% of theory

R_{f} value: 0.65 (silica gel; ethyl acetate/ethanol=9:1+a few drops of ammonia)

C₂₆H₂₈N₆O₆ (520.55)

Mass spectrum: (M+H)⁺=521

### EXAMPLE 24

### N-(n-hexyloxycarbonyl)-4-hydroxy-3-{[7-(pyrrolidin-1-yl-carbonyl)-quinazolin-4-yl]aminomethyl}-benzamidine

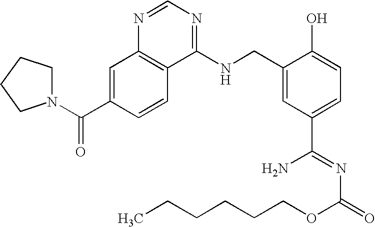

0.390 g (1.00 mmol) of 4-hydroxy-3-{[7-(pyrrolidin-1-yl-carbonyl)-quinazolin-4-yl]aminomethyl}-benzamidine are suspended in 15 ml methylene chloride, combined with 0.289 g (1.08 mmol) of 4-n-hexyloxycarbonyloxy-nitrobenzene and 0.70 ml (5.0 mmol) of triethylamine and heated to boiling for 2.5 h. The reaction mixture is washed with water and saturated sodium chloride solution, dried over magnesium sulphate and concentrated. After purification by chromatography the title compound is obtained (silica gel, ethyl acetate/methanol 100:0→80:20).

Yield: 20 mg (4% of theory)

R_{f} value: 0.63 (silica gel; ethyl acetate/ethanol=9:1)

C₂₈H₃₄N₆O₄ (518.621)

Mass spectrum: (M+H)⁺=519

### EXAMPLE 25

### 3-{[7-(N-ethyl-N-methyl-aminocarbonyl)-(quinazolin-4-yl]aminomethyl}-4-hydroxy-benzamidine-hydrochloride

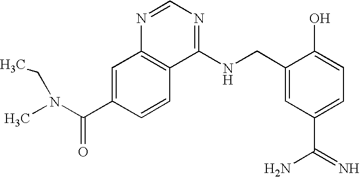

Prepared analogously to Example 2i from 4-benzyloxy-3-{[7-(thiazolidin-3-yl-carbonyl)-quinazolin-4-yl]aminomethyl}-benzamidine-dihydrochloride, palladium on activated charcoal and hydrogen in methanol and subsequent purification by chromatography.

Yield: 17% of theory

R_{f} value: 0.50 (Reversed phase RP8; 5% sodium chloride solution/methanol=4:6)

C₂₀H₂₂N₆O₂×2 HCl (378.44/451.36)

Mass spectrum: (M+H)⁺=379

### EXAMPLE 26

### 4-hydroxy-3-{[7-(thiazolidin-3-yl-carbonyl)-quinazolin-4-yl]aminomethyl}-benzamidine-dihydrochloride

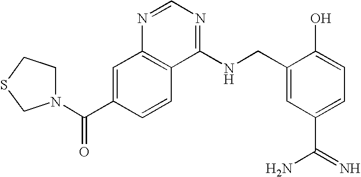

0.210 g (0.367 mmol) of 4-benzyloxy-3-{[7-(thiazolidin-3-yl-carbonyl)-quinazolin-4-yl]aminomethyl}-benzamidine-dihydrochloride and 0.435 g (2.94 mmol) of pentamethylbenzene are heated to 60° C. in 5 ml trifluoroacetic acid for 18 h and after the further addition of 80 mg pentamethylbenzene, heated for another 3 h at 70° C. Then the mixture is evaporated down and purified through a silica gel column (methylene chloride/methanol 90:10→80:20).

Yield: 91% of theory

R_{f} value: 0.55 (Reversed phase RP8; 5% sodium chloride solution/methanol=4:6)

C₂₀H₂₀N₆O₂S×2 HCl (408.49/481.41)

Mass spectrum: (M+H)⁺=409
(M+H)⁻=407

### EXAMPLE 27

### 4-benzyloxy-3-{[2-methyl-7-(pyrrolidin-1-yl-carbonyl)-quinazolin-4-yl]aminomethyl}-benzamidine-hydrochloride

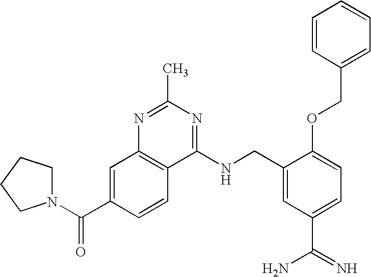

Prepared analogously to Example 2h from 4-benzyloxy-3-{[2-methyl-7-(pyrrolidin-1-yl-carbonyl)-quinazolin-4-yl]aminomethyl}-benzonitrile and hydrochloric acid/ammonium carbonate in ethanol.

Yield: 25% of theory

R_{f} value: 0.11 (silica gel; dichloromethane/ethanol=4:1)

C₂₉H₃₀N₆O₂×HCl (494.60/531.06)

Mass spectrum: (M+H)⁺=495

### EXAMPLE 28

### 4-hydroxy-3-{[2-methyl-7-(pyrrolidin-1-yl-carbonyl)-quinazolin-4-yl]aminomethyl}-benzamidine-hydrochloride

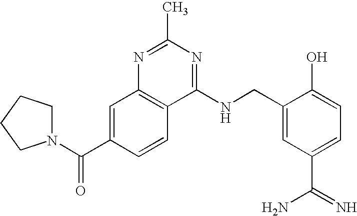

Prepared analogously to Example 2i from 4-benzyloxy-3-{[2-methyl-7-(pyrrolidin-1-yl-carbonyl)-quinazolin-4-yl]aminomethyl}-benzamidine-hydrochloride, palladium on activated charcoal and hydrogen in methanol.

Yield: 96% of theory

R_{f} value: 0.57 (Reversed phase RP8; 5% sodium chloride solution/methanol 4:6)

C₂₂H₂₄N₆O₂×HCl (404.48/440.94)

Mass spectrum: (M+H)⁺=405
(M−H)⁻=403

### EXAMPLE 29

### 4-hydroxy-3-{[6-chloro-7-(pyrrolidin-1-yl-carbonyl)-quinazolin-4-yl]aminomethyl}-benzamidine-acetate

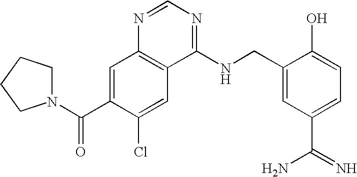

### a. dimethyl 2-amino-5-chloro-terephthalate

104 g (0.50 mol) dimethyl aminoterephthalate are dissolved in 750 ml methylene chloride and at 0° C. combined with 77 ml (0.96 mol) sulphuryl chloride. Then the mixture is heated to boiling for one hour, combined with 200 ml diethyl ether, and the precipitate formed is filtered off.

Yield: 23% of theory

R_{f} value: 0.45 (silica gel; petroleum ether/ethyl acetate=6:4)

C₁₀H₁₀ClNO₄ (243.65)

Mass spectrum: (M+H)⁺=244/246 (chlorine isotope)

### b. 4-hydroxy-3-{[6-chloro-7-(pyrrolidin-1-yl-carbonyl)-quinazolin-4-yl]aminomethyl}-benzamidine-acetate

The title compound is prepared from dimethyl 2-amino-5-chloro-terephthalate after saponification with sodium hydroxide solution analogously to Example 13 and the synthesis sequence analogous to Examples 8a, 8b, 8c, 8h, 2h and final debenzylation with palladium/charcoal in methanol analogously to Example 2i and purification by chromatography (silica gel: methylene chloride/ethanol 9:1-7:3+1% glacial acetic acid).

Yield: 13% of theory (last step)

R_{f} value: 0.65 (silica gel; methylene chloride/ethanol 7:3+1% glacial acetic acid)

C₂₁H₂₁ClN₆O₂×C₂H₄O₂ (424.90/484.95)

Mass spectrum: (M+H)⁺=425/427 (chlorine isotope)

(M−H)⁻=423/425 (chlorine isotope)

### EXAMPLE 30

### 5-{[6-methyl-7-(pyrrolidin-1-yl-carbonyl)-quinazolin-4-yl]aminomethyl}-thiophene-2-amidine-hydrochloride

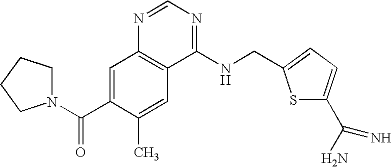

Prepared analogously to Example 2h from 5-{[6-methyl-7-(pyrrolidin-1-yl-carbonyl)-quinazolin4-yl]aminomethyl}-thiophene-2-nitrile and hydrochloric acid/ammonium carbonate in ethanol.

Yield: 56% of theory

C₂₀H₂₂N₆OS×HCl (394.50/430.96)

Mass spectrum: (M+H)⁺=395
(M+Cl)⁻=429/431 (chlorine isotope)
(M−H)⁻=393

### EXAMPLE 31

### 4-hydroxy-3-{2-phenyl-1-[7-(pyrrolidin-1-yl-carbonyl)-quinolin-4-ylamino]-ethyl}-benzamidine-hydrochloride

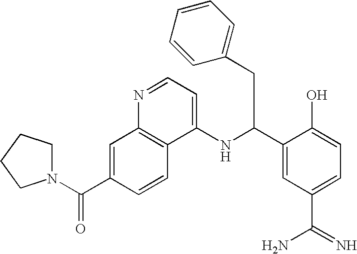

Prepared analogously to Example 2i from 4-benzyloxy-3-{2-phenyl-1-[7-(pyrrolidin-1-yl-carbonyl)-quinolin-4-ylamino]-ethyl}-benzamidine-hydrochloride, palladium on activated charcoal and hydrogen in methanol.

Yield: 75% of theory

R_{f} value: 0.69 (silica gel; acetonitrile/chloroform/water/formic acid 75:20:10:15)

C₂₉H₂₉N₅O₂×HCl (479.58/516.05)

Mass spectrum: (M+H)⁺=480

### EXAMPLE 32

### N-benzoyl-4-hydroxy-3-{[6-methyl-7-(pyrrolidin-1-yl-carbonyl)-quinazolin-4-yl]aminomethyl}-benzamidine

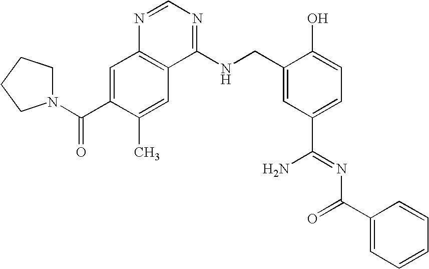

### a. N-benzoyl-4-benzyloxy-3-{[6-methyl-7-(pyrrolidin-1-yl-carbonyl)-quinazolin-4-yl]aminomethyl}-benzamidine

Prepared analogously to Example 20 from 4-benzyloxy-3-{[6-methyl-7-(pyrrolidin-1-yl-carbonyl)-quinazolin-4-yl]aminomethyl}-benzamidine, 4-nitrophenyl-benzoate and triethylamine in N,N-dimethylformamide.

Yield: 7.1%

R_{f} value: 0.45 (silica gel; ethyl acetate/ethanol 9:1+0.5% ammonia solution)

C₂₈H₂₆N₆O₃ (494.56).

### b. N-benzoyl-4-hydroxy-3-{[6-methyl-7-(pyrrolidin-1-yl-carbonyl)-quinazolin-4-yl]aminomethyl}-benzamidine

Prepared analogously to Example 2i from N-benzoyl-4-benzyloxy-3-{[6-methyl-7-(pyrrolidin-1-yl-carbonyl)-quinazolin-4-yl]aminomethyl}-benzamidine, palladium on activated charcoal and hydrogen in methanol.

Yield: 34% of theory

R_{f} value: 0.36 (silica gel; ethyl acetate/ethanol 9:1+0.5% ammonia solution)

C₂₉H₂₈N₆O₃ (508.56)

Mass spectrum: (M+H)⁺=509

### EXAMPLE 33

### N-benzoyl-3-{[6-chloro-7-(pyrrolidin-1-yl-carbonyl)-quinazolin-4-yl]aminomethyl}4-hydroxy-benzamidine

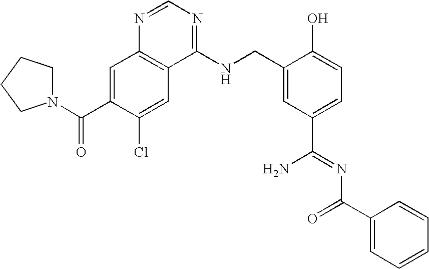

Prepared analogously to Example 24 from 4-benzyloxy-3-{[6-chloro-7-(pyrrolidin-1-yl-carbonyl)-quinazolin-4-yl]aminomethyl}-benzamidine, 4-nitrophenyl-benzoate and triethylamine in dichloromethane and subsequently reacting analogously to Example 2i with palladium on activated charcoal/hydrogen in methanol.

Yield: 26% of theory over 2 steps

R_{f} value: 0.71 (silica gel; dichloromethane/ethanol 9:1)

C₂₈H₂₅ClN₆O₃ (529.00)

Mass spectrum: (M+H)⁺=529/531 (chlorine isotope)

### EXAMPLE 34

### 3-{[6-chloro-7-(pyrrolidin-1-yl-carbonyl )-quinazolin in-4-yl]aminomethyl}-N -(n-hexyloxycarbonyl)-4-hydroxy-benzamidine

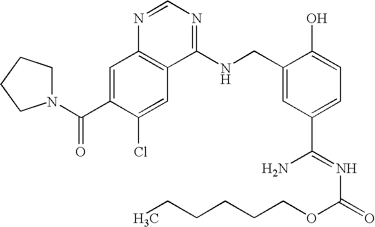

### a. 4-benzyloxy-3-{[6-chloro-7-(pyrrolidin-1-yl-carbonyl )-quinazolin-4-yl]aminomethyl}-N-(n-hexyloxycarbonyl)-benzamidine

Prepared analogously to Example 24 from 4-benzyloxy-3-{[6-chloro-7-(pyrrolidin-1-yl-carbonyl)-quinazolin-4-yl]aminomethyl}-benzamidine, O-(n-hexyl)-O′-(p-nitrophenyl)-carbonate and triethylamine in dichloromethane.

Yield: 67%

R_{f} value: 0.75 (silica gel; dichloromethane/ethanol 9:1)

Mass spectrum: (M+H)⁺=643/645 (chlorine isotope)

C₃₅H₃₉ClN₆O₄ (643,19).

### b. 3-{[6-chloro-7-(pyrrolidin-1-yl-carbonyl)-quinazolin-4-yl]aminomethyl}-N-(n-hexyloxycarbonyl)-4-hydroxy-benzamidine

Prepared analogously to Example 2i from 4-benzyloxy-3-{[6-chloro-7-(pyrrolidin-1-yl-carbonyl)-quinazolin-4-yl]aminomethyl}-N-(n-hexyloxycarbonyl)-benzamidine, palladium on activated charcoal and hydrogen in methanol.

Yield: 51% of theory

R_{f} value: 0.70 (silica gel; ethyl acetate/ethanol 9:1)

C₂₈H₃₃ClN₆O₄ (553.07)

Mass spectrum: (M+H)⁺=553/555 (chlorine isotope)

### EXAMPLE 35

### 2-amidino-5-{[7-(2,5-dihydropyrrol-1-yl-carbonyl)-quinazolin-4-yl]aminomethyl}-thiophene-hydrochloride

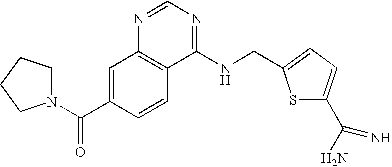

Prepared analogously to Example 2h from 2-cyano-5-{[7-(2,5-dihydropyrrol-1-yl-carbonyl)-quinazolin-4-yl]aminomethyl}-thiophene and hydrogen chloride/ammonium carbonate in ethanol.

Yield: 38% of theory

R_{f} value: 0.1 (silica gel; ethyl acetate/ethanol 8:2)

C₁₉H₁₈N₆OS*HCl (414.92/378.46)

Mass spectrum: (M+H)⁺=379

### EXAMPLE 36

### 3-{[7-(2,5-dihydropyrrol-1-yl-carbonyl)-quinazolin-4-yl]aminomethyl}-benzamidine-hydrochloride

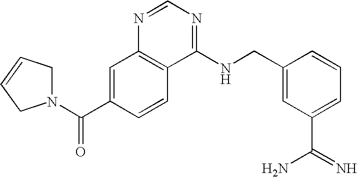

Prepared analogously to Example 2h from 3-{[7-(2,5-dihydropyrrol-1-yl-carbonyl)-quinazolin-4-yl]aminomethyl}-benzonitrile and hydrogen chloride/ammonium carbonate in ethanol.

Yield: 91% of theory

R_{f} value: 0.07 (silica gel; dichloromethane/ethanol 8:2)

C₂₁H₂₀N₆O*HCl (408.89/372.43)

Mass spectrum: (M+H)⁺=373

### EXAMPLE 37

### 3-{[7-(Pyrrolidin-1-yl-carbonyl)-quinazolin-4-yl]aminomethyl}-benzamidine-hydrochloride

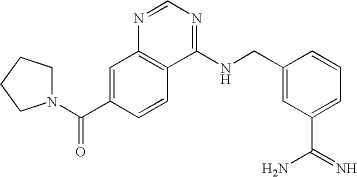

150 mg (0.37 mmol) of 3-{[7-(2,5-dihydropyrrol-1-yl-carbonyl)-quinazolin-4-yl]aminomethyl}-benzamidine-hydrochloride are dissolved in 5.0 ml of methanol, combined with 15 mg platinum dioxide and hydrogenated for 2.5 hours at 3 bar under a hydrogen atmosphere. After filtration the solvent is eliminated in vacuo, the residue is taken up in ethanol and filtered through silica gel. The filtrate is freed from solvent in vacuo.

Yield: 50 mg of yellow crystals (33% of theory)

R_{f} value: 0.21 (Reversed phase RP8; 5% sodium chloride solution/methanol 3:2)

C₂₁H₂₂N₆O*HCl (374.45/410.91)

Mass spectrum: (M+H)⁺=375

### EXAMPLE 38

### 4-hydroxy-3-{2-phenyl-1-[7-(pyrrolidin-1-yl-carbonyl)-quinolin-4-ylamino]-ethyl}-benzylamine

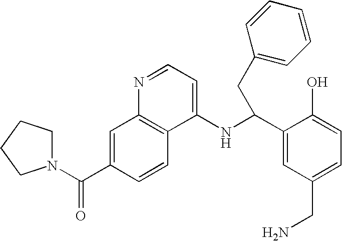

Prepared analogously to Example 2i from 4-benzyloxy-3-{2-phenyl-1-[7-(pyrrolidin-1-yl-carbonyl)-quinolin-4-ylamino]-ethyl}-benzonitrile, palladium on activated charcoal and hydrogen in methanol.

Yield: 91% of theory

R_{f} value: 0.42 (silica gel; dichloromethane/methanol 8:2+0.1% ammonia solution)

C₂₉H₃₀N₄O₂ (466.59)

Mass spectrum: (M+H)⁺=467

### EXAMPLE 39

### 3-{[7-(3-amino-piperidin-1-yl-carbonyl)-quinazolin-4-yl]aminomethyl}-benzamidine

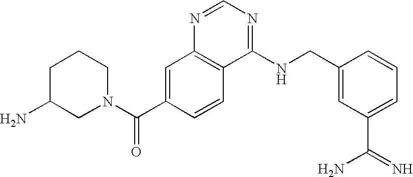

Prepared analogously to Example 8h from 4-chloro-7-(3-tert.-butoxycarbonylamino-piperidin-1-yl-carbonyl)-quinazoline, 3-aminomethyl-benzamidine and N,N-diisopropylethylamine in dimethylformamide at ambient temperature. Then in order to cleave the Boc, after eliminating the solvent in vacuo, the residue is treated with a mixture of dichloromethane/trifluoroacetic acid/distilled water 30:63:7 over 2 days. Volatile constituents are then eliminated in vacuo.

HPLC-MS results:

retention time: 2.31 min

C₂₂H₂₅N₇O (403.49)

Mass spectrum: (M+H)⁺=404

### EXAMPLE 40

### 3-{[7-(2-aminomethyl-piperidin-1-yl-carbonyl )-quinazolin-4-yl]aminomethyl}-benzamidine

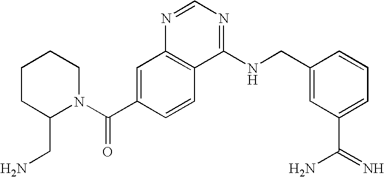

Prepared analogously to Example 8h from 4-chloro-7-(2-tert.-butoxycarbonylaminomethyl-piperidin-1-yl-carbonyl)-quinazoline, 3-aminomethyl-benzamidine and diisopropylethylamine in dimethylformamide at ambient temperature and subsequent Boc cleaving with trifluoroacetic acid analogously to Example 39.

HPLC-MS results:

retention time: 2.46 min

C₂₃H₂₇N₇O (417.51)

Mass spectrum: (M+H)⁺=418

### EXAMPLE 41

### 3-{[7-(azetidin-1-yl-carbonyl)-quinazolin-4-yl]aminomethyl}-benzamidine

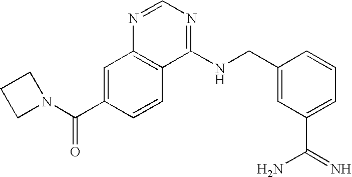

Prepared analogously to Example 8h from 4-chloro-7-(azetidin-1-yl-carbonyl)-quinazoline, 3-aminomethyl-benzamidine and diisopropylethylamine in dimethylformamide at ambient temperature.

HPLC-MS results:

retention time: 2.52 min

C₂₀H₂₀N₆O (360.42)

Mass spectrum: (M+H)⁺=361

### EXAMPLE 42

### 3-{[7-(3-aminomethyl-piperidin-1-yl-carbonyl)-quinazolin-4-yl]aminomethyl}-benzamidine

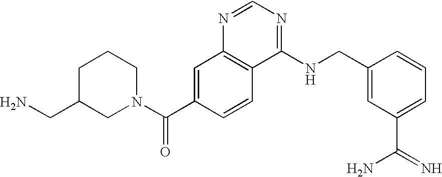

Prepared analogously to Example 8h from 4-chloro-7-(3-tert.-butoxycarbonylaminomethyl-piperidin-1-yl-carbonyl)-quinazoline, 3-aminomethyl-benzamidine and diisopropylethylamine in dimethylformamide at ambient temperature and subsequent Boc cleaving with trifluoroacetic acid analogously to Example 39.

HPLC-MS results:

retention time: 2.41 min

C₂₃H₂₇N₇O (417.51)

Mass spectrum: (M+H)⁺=418

### EXAMPLE 43

### 3-{[7-(2,5-dihydropyrrol-1-yl-carbonyl)-quinazolin-4-yl]aminomethyl}-benzamidine

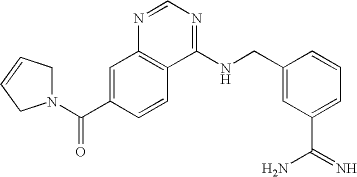

Prepared analogously to Example 8h from 4-chloro-7-(2,5-dihydropyrrol-1-yl-carbonyl)-quinazoline, 3-aminomethyl-benzamidine and diisopropylethylamine in dimethylformamide at ambient temperature.

HPLC-MS results:

retention time: 2.64 min

C₂₁H₂₀N₆O (372.43)

Mass spectrum: (M+H)⁺=373

### EXAMPLE 44

### 3-{[7-(piperidin-1-yl-carbonyl)-quinazolin-4-yl]aminomethyl}-benzamidine

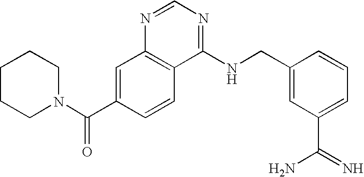

Prepared analogously to Example 8h from 4-chloro-7-(piperidin-1-yl-carbonyl)-quinazoline, 3-aminomethyl-benzamidine and diisopropylethylamine in dimethylformamide at ambient temperature.

HPLC-MS results:

retention time: 2.77 min

C₂₂H₂₄N₆O (388.47)

Mass spectrum: (M+H)⁺=389

### EXAMPLE 45

### 3-{(7-diethylaminocarbonyl-quinazolin-4-yl)aminomethyl}-benzamidine

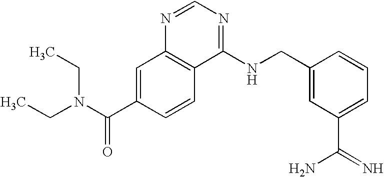

Prepared analogously to Example 8h from 4-chloro-7-diethylaminocarbonyl-quinazoline, 3-aminomethyl-benzamidine and diisopropylethylamine in dimethylformamide at ambient temperature.

HPLC-MS results:

retention time: 2.70 min

C₂₁H₂₄N₆O (376.46)

Mass spectrum: (M+H)⁺=377

### EXAMPLE 46

### 3-{[7-(3-dimethylaminoazetidin-1-yl-carbonyl)-quinazolin-4-yl]aminomethyl}-benzamidine

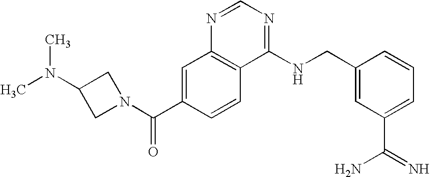

Prepared analogously to Example 8h from 4-chloro-7-(3-dimethylaminoazetidin-1-yl-carbonyl)-quinazoline, 3-aminomethyl-benzamidine and diisopropylethylamine in dimethylformamide at ambient temperature.

HPLC-MS results:

retention time: 2.32 min

C₂₂H₂₅N₇O (403.49)

Mass spectrum: (M+H)⁺=404

### EXAMPLE 47

### 3-{[7-(N-cyclohexyl-N-methyl-aminocarbonyl)-quinazolin-4-yl]aminomethyl}-benzamidine

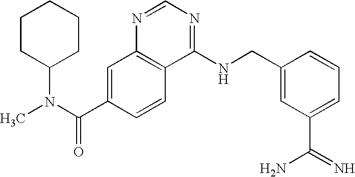

Prepared analogously to Example 8h from 4-chloro-7-(N-cyclohexyl-N-methyl-aminocarbonyl)-quinazoline, 3-aminomethyl-benzamidine and diisopropylethylamine in dimethylformamide at ambient temperature.

HPLC-MS results:

retention time: 3.15 min

C₂₄H₂₈N₆O (416.53)

Mass spectrum: (M+H)⁺=417

### EXAMPLE 48

### 3-{[7-(N-benzyl-N-methyl-aminocarbonyl)-(quinazolin-4-(yl]aminomethyl}-benzamidine

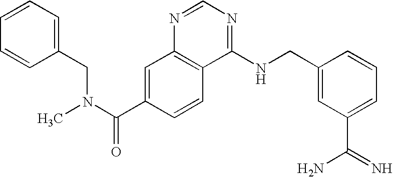

Prepared analogously to Example 8h from 4-chloro-7-(N-benzyl-N-methyl-aminocarbonyl)-quinazoline, 3-aminomethyl-benzamidine and diisopropylethylamine in dimethylformamide at ambient temperature.

HPLC-MS results:

retention time: 3.11 min

C₂₅H₂₄N₆O (424.51)

Mass spectrum: (M+H)⁺=425

### EXAMPLE 49

### 3-{[7-(3-amino-pyrrolidin-1-yl-carbonyl)-quinazolin-4-yl]aminomethyl}-benzamidine

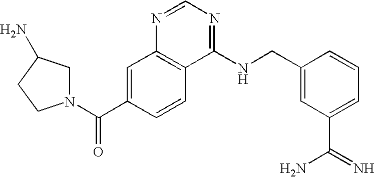

Prepared analogously to Example 8h from 4-chloro-7-(3-tert.-butoxycarbonylamino-pyrrolidin-1-yl-carbonyl)-quinazoline, 3-aminomethyl-benzamidine and diisopropylethylamine in dimethylformamide at ambient temperature and subsequent Boc cleaving with trifluoroacetic acid analogously to Example 39.

HPLC-MS results:

retention time: 2.31 min

C₂₁H₂₃N₇O (389.46)

Mass spectrum: (M+H)⁺=390

### EXAMPLE 50

### 3-{[7-(pyrrolidin-1-yl-carbonyl)-6-chloro-quinazolin-4-yl]aminomethyl}-4-hydroxy-benzylamine

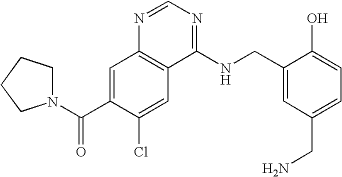

### a. 3-{[7-(pyrrolidin-1-yl-carbonyl)-6-chloro-quinazolin-4-yl]aminomethyl}-4-benzyloxy-benzylamine

496 mg (1.00 mmol) of 3-{[7-(2,5-dihydropyrrol-1-yl-carbonyl)-6-chloro-quinazolin-4-yl]aminomethyl}-4-benzyloxy-benzonitrile are combined with 500 mg Randy nickel in 75 ml of methanolic ammonia solution and treated with hydrogen for 7.5 hours at 5 atom pressure. Then the mixture is filtered and the solvent is eliminated in vacuo. The residue is treated with 20 ml dichloromethane/diethyl ether 1:1 and the crystalline solid is filtered off.

Yield: 210 mg (42% of theory)

R_{f} value: 0.15 (silica gel; dichloromethane/ethanol 9:1+1% acetic acid)

C₂₈H₂₈CIN₅O₂ (502.02)

Mass spectrum: (M+H)⁺=502/504 (chlorine isotope)

### b. 3-{[7-(pyrrolidin-1-yl-carbonyl)-6-chloro-quinazolin-4-yl]aminomethyl}-4-hydroxy-benzylamin-hydrochloride

75 mg (0.15 mmol) of 3-{[7-(pyrrol-1-yl-carbonyl)-6-chloro-quinazolin-4-yl]aminomethyl}-4-benzyloxy-benzylamine are combined with 180 mg pentamethylbenzene in 3 ml trifluoroacetic acid and heated to 60° C. for 6 hours under a nitrogen atmosphere. Then the mixture is stirred for 16 hours at ambient temperature, volatile constituents are eliminated in vacuo and the residue is combined with 50 ml ice water. The precipitate formed is filtered off and the filtrate is evaporated down in vacuo. The residue is taken up twice in toluene and once in diethyl ether and in each case evaporated down in vacuo. The residue remaining is dissolved in 2 ml absolute ethanol and treated with ethereal hydrochloric acid. The resulting precipitate is filtered off, treated with 30 ml of solvent mixture comprising petroleum ether/diethyl ether/ethyl acetate 1:10:1 and the crystalline solid is filtered off.

Yield: 24 mg (36% of theory)

R_{f} value: 0.15 (Reversed phase RP8; 5% sodium chloride solution/methanol 3:2)

C₂₁H₂₂ClN₅O₂×HCl (448.36/411.89)

Mass spectrum: (M+H)⁺=412/414 (chlorine isotope)

The Examples that follow describe the preparation of pharmaceutical formulations which contain as active substance any desired compound of general formula (I):

### EXAMPLE I

Dry ampoule containing 75 mg of active substance per 10 ml

Composition:

| | | |
|---|---|---|
| Active substance | 75.0 | mg |
| Mannitol | 50.0 | mg |
| water for injections | ad 10.0 | ml |

Preparation:

Active substance and mannitol are dissolved in water. After packaging the solution is freeze-dried. To produce the solution ready for use for injections, the product is dissolved in water.

### EXAMPLE II

Dry ampoule containing 35 mg of active substance per 2 ml

Composition:

| | | |
|---|---|---|
| Active substance | 35.0 | mg |
| Mannitol | 100.0 | mg |
| water for injections | ad 2.0 | ml |

Preparation:

Active substance and mannitol are dissolved in water. After packaging, the solution is freeze-dried.

To produce the solution ready for use for injections, the product is dissolved in water.

### EXAMPLE III

Tablet containing 50 mg of active substance

Composition:

| | |
|---|---|
| (1) Active substance | 50.0 mg |
| (2) Lactose | 98.0 mg |
| (3) Maize starch | 50.0 mg |
| (4) Polyvinylpyrrolidone | 15.0 mg |
| (5) Magnesium stearate | 2.0 mg |
| | 215.0 mg |

Preparation:

(1), (2) and (3) are mixed together and granulated with an aqueous solution of (4). (5) is added to the dried granulated material. From this mixture tablets are pressed, biplanar, faceted on both sides and with a dividing notch on one side.

Diameter of the tablets: 9 mm.

### EXAMPLE IV

Tablet containing 350 mg of active substance

Composition:

| | |
|---|---|
| (1) Active substance | 350.0 mg |
| (2) Lactose | 136.0 mg |
| (3) Maize starch | 80.0 mg |
| (4) Polyvinylpyrrolidone | 30.0 mg |
| (5) Magnesium stearate | 4.0 mg |
| | 600.0 mg |

Preparation:

(1), (2) and (3) are mixed together and granulated with an aqueous solution of (4). (5) is added to the dried granulated material. From this mixture tablets are pressed, biplanar, faceted on both sides and with a dividing notch on one side.

Diameter of the tablets: 12 mm.

### EXAMPLE V

Capsules containing 50 mg of active substance

Composition:

| | |
|---|---|
| (1) Active substance | 50.0 mg |
| (2) Dried maize starch | 58.0 mg |
| (3) Powdered lactose | 50.0 mg |
| (4) Magnesium stearate | 2.0 mg |
| | 160.0 mg |

Preparation:

(1) is triturated with (3). This trituration is added to the mixture of (2) and (4) with vigorous mixing.

This powder mixture is packed into size 3 hard gelatine capsules in a capsule filling machine.

### EXAMPLE VI

Capsules containing 350 mg of active substance

Composition:

| | |
|---|---|
| (1) Active substance | 350.0 mg |
| (2) Dried maize starch | 46.0 mg |
| (3) Powdered lactose | 30.0 mg |
| (4) Magnesium stearate | 4.0 mg |
| | 430.0 mg |

Preparation:

(1) is triturated with (3). This trituration is added to the mixture of (2) and (4) with vigorous mixing.

This powder mixture is packed into size 0 hard gelatine capsules in a capsule filling machine.

### EXAMPLE VII

Suppositories containing 100 mg of active substance

1 suppository contains:

| | |
|---|---|
| Active substance | 100.0 mg |
| Polyethyleneglycol (M.W. 1500) | 600.0 mg |
| Polyethyleneglycol (M.W. 6000) | 460.0 mg |
| Polyethylenesorbitan monostearate | 840.0 mg |
| | 2,000.0 mg |

Preparation

The polyethyleneglycol is melted together with polyethylenesorbitan monostearate. At 40° C. the ground active substance is homogeneously dispersed in the melt. It is cooled to 38° C. and poured into slightly chilled suppository moulds.

1. A compound of general formula 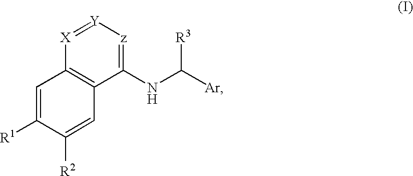
wherein
X denotes a nitrogen atom or a methyne group,
Y denotes a methyne group optionally substituted by a C₁₋₃-alkyl or amino group, or a nitrogen atom,
Z denotes a nitrogen atom or a methyne group,
R¹ denotes an amino, C₁₋₅-alkylamino, C₃₋₇-cycloalkylamino or phenyl-C₁₋₃-alkyl-amino group which may be substituted in each case at the amino nitrogen atom by a phenylcarbonyl or phenylsulphonyl group or by a C₁₋₃-alkyl or C₁₋₃-alkyl-carbonyl group optionally substituted in the alkyl moiety by a carboxy group, a group which may be converted in vivo into a carboxy group, an amino, C₁₋₃-alkylamino or di-(C₁₋₃-alkyl)-amino group, a di-(C₁₋₅-alkyl)amino or N—(C₃₋₇-cycloalkyl)-C₁₋₅-alkylamino group, wherein the C₁₋₅-alkyl moiety with the exception of the 1-position may be substituted in each case by a hydroxy, C₁₋₃-alkoxy, amino, C₁₋₃-alkyl-amino or di-(C₁₋₃-alkyl)-amino group,
a 4- to 7-membered cycloalkyleneimino group, while a methylene group which is not directly adjacent to the imino group, may be substituted by a hydroxy, C₁₋₃-alkoxy, amino, C₁₋₃-alkyl-amino or di-(C₁₋₃-alkyl)-amino group,
a 4- to 7-membered cycloalkyleneiminocarbonyl or cycloalkyleneiminosulphonyl group, while
the cycloalkyleneimino moiety may be substituted by a C₁₋₃-alkyl, amino-C₁₋₃-alkyl, C₁₋₃-alkylamino-C₁₋₃-alkyl, di-(C₁₋₃-alkyl)-amino-C₁₋₃-alkyl, aminocarbonyl, C₁₋₃-alkylamino-carbonyl, N-(C₃₋₇-cycloalkyl)-C₁₋₅-alkyl-aminocarbonyl, N-(phenyl-C₁₋₃-alkyl)-C₁₋₅-alkylaminocarbonyl or di-(C₁₋₃-alkyl)-aminocarbonyl group or
a methylene group not adjacent to the imino group may be substituted by a hydroxy, benzyloxy, amino, C₁₋₃-alkylamino or di-(C₁₋₃-alkyl)-amino group and/or
a methylene group in the 3 position of a 5-, 6- or 7-membered cycloalkyleneimino group may be replaced by a sulphur atom or by a sulphinyl or sulphonyl group or
a methylene group in the 4 position of a 6- or 7-membered cycloalkyleneimino group may be replaced by an oxygen or sulphur atom or by a —NH—, —N(C₂₋₃-alkanoyl)-, sulphinyl or sulphonyl group and/or
a —CH₂—CH₂— group in a 5- to 7-membered cycloalkyleneimino group may be replaced by a —NH—CO— group,
a 2,5-dihydropyrrol-1-yl-carbonyl or 1,2,5,6-tetrahydropyridin-1-yl-carbonyl group optionally substituted by a C₁₋₃-alkyl, amino-C₁₋₃-alkyl, C₁₋₃-alkylamino-C₁₋₃-alkyl, di-(C₁₋₃-alkyl)-amino-C₁₋₃-alkyl, aminocarbonyl, C₁₋₃-alkylamino-carbonyl, N—(C₃₋₇-cycloalkyl)-C₁₋₅-alkylaminocarbonyl, N-(phenyl-C₁₋₃-alkyl)-C₁₋₅-alkylaminocarbonyl or di-(C₁₋₃-alkyl)-aminocarbonyl group,
an aminosulphonyl or aminocarbonyl group optionally substituted by one or two C₁₋₃-alkyl, phenyl-C₁₋₃-alkyl or C₃₋₇-cycloalkyl groups, while the substituents may be identical or different,
a straight-chain or branched C₁₋₅-alkylcarbonyl group,
a C₃₋₇-cycloalkyl-carbonyl group, while
the methylene group in the 3 or 4 position in a C₅₋₇-cycloalkyl-carbonyl group may be replaced by a —NH— group, while
the hydrogen atom of the —NH— group may be replaced by a C₁₋₃-alkyl, C₁₋₃-alkyl-carbonyl, phenylcarbonyl or phenylsulphonyl group,
a phenylcarbonyl or heteroarylcarbonyl group or
a C₁₋₃-alkyl group optionally substituted by an amino, C₁₋₃-alkylamino, di-(C₁₋₃-alkyl)-amino, hydroxy, phenyl or a 4- to 7-membered cycloalkyleneimino group, while
the phenyl substituents may be substituted by a fluorine, chlorine or bromine atom, by a trifluoromethyl, C₁₋₃-alkyl or C₁₋₃-alkoxy group,
R² denotes a hydrogen, fluorine, chlorine or bromine atom,
a C₁₋₃-alkyl group wherein the hydrogen atoms may be wholly or partly replaced by fluorine atoms, or
a C₂₋₃-alkenyl, C₂₋₃-alkynyl, hydroxy, C₁₋₃-alkoxy or trifluoromethoxy group,
R³ denotes a hydrogen atom,
a straight-chain or branched C₁₋₆-alkyl group which is optionally substituted by a hydroxy, C₁₋₃-alkyloxy, carboxy, C₁₋₃-alkoxy-carbonyl, amino, C₁₋₃-alkylamino, di-(C₁₋₃-alkyl)-amino, C₁₋₃-alkyl-carbonylamino, C₁₋₅-alkoxy-carbonylamino or phenyl-C₁₋₃-alkoxy-carbonylamino group,
a methyl or ethyl group which are substituted in each case
by a phenyl or heteroaryl group which are substituted optionally in each case by a hydroxy, C₁₋₄-alkyloxy, benzyloxy, hydroxycarbonyl-C₁₋₃-alkoxy, C₁₋₃-alkyloxy-carbonyl-C₁₋₃-alkyloxy, aminocarbonyl-C₁₋₃-alkyloxy, C₁₋₃-alkyl-aminocarbonyl-C₁₋₃-alkyloxy, di-(C₁₋₃-alkyl)-aminocarbonyl-C₁₋₃-alkyloxy, carboxy, C₁₋₃-alkyloxy-carbonyl group,
by a 1-H-pyridonyl or 1-(C₁₋₃-alkyl)-pyridonyl group,
by a 4- to 7-membered cycloalkyleneimino group or
by a 4- to 7-membered cycloalkyl group wherein one or two methylene groups separated from one another by at least a methylene group are each replaced by an oxygen or sulphur atom or by a —NH— or —N(C₁₋₃-alkyl)-group and wherein, if the cycloalkyl group contains an —NH— or an —N(C₁₋₃-alkyl)-group, a methylene group adjacent to the nitrogen atom and, if the cycloalkyl group contains a total of two —NH or —N(C₁₋₃-alkyl)-groups, a methylene group adjacent to both nitrogen atoms may be replaced by a carbonyl group, or
a phenyl or heteroaryl group which may be substituted in each case by a hydroxy, C₁₋₄-alkyloxy, benzyloxy, hydroxycarbonyl-C₁₋₃-alkoxy, C₁₋₃-alkyloxy-carbonyl-C₁₋₃-alkyloxy, aminocarbonyl-C₁₋₃-alkyloxy, C₁₋₃-alkylaminocarbonyl-C₁₋₃-alkyloxy, di-(C₁₋₃-alkyl)-aminocarbonyl-C₁₋₃-alkyloxy, carboxy, C₁₋₃-alkyloxy-carbonyl group, and
Ar denotes a phenyl group substituted by the groups R⁴, R⁵ and R⁶, where
R⁴ denotes a cyano group,
an amidino group optionally substituted by one or two hydroxy, C₁₋₃-alkyl, C₁₋₈-alkyl-carbonyl, C₁₋₈-alkoxy-carbonyl or benzoyl groups,
an amino-C₁₋₃-alkyl, C₁₋₃-alkylamino-C₁₋₃-alkyl or di-(C₁₋₃-alkyl)amino-C₁₋₃-alkyl group or
a group of formula 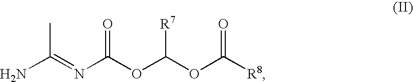
wherein R⁷ denotes a hydrogen atom or a C₁₋₃-alkyl group and
R⁸ denotes a C₁₋₃-alkyl group,
R⁵ denotes a hydrogen, fluorine, chlorine or bromine atom or a trifluoromethyl, C₁₋₃-alkyl, hydroxy, hydroxy-C₁₋₃-alkyl, C₁₋₃-alkoxy, benzyloxy, C₁₋₃-alkoxy-C₁₋₃-alkyl, amino, C₁₋₃-alkylamino or di-(C₁₋₃-alkyl)amino group and
R⁶ denotes a hydrogen, fluorine, chlorine or bromine atom or a C₁₋₃-alkyl group,
or a thienylene, thiazolylene, pyridinylene, pyrimidinylene, pyrazinylene or pyridazinylene group optionally substituted in the carbon skeleton by the groups R⁴ and R⁵, where R⁴ and R⁵ are as hereinbefore defined,
while, unless otherwise mentioned, the expression a “heteroaryl group” refers to a monocyclic 5- or 6-membered heteroaryl group optionally substituted in the carbon skeleton by a C₁₋₃-alkyl, carboxy, C₁₋₃-alkoxy-carbonyl or C₁₋₃-alkoxy-carbonylamino group, while
the 6-membered heteroaryl group contains one, two or three nitrogen atoms and
the 5-membered heteroaryl group denotes an imino group optionally substituted by a C₁₋₃-alkyl or phenyl-C₁₋₃-alkyl group, an oxygen or sulphur atom or
an imino group optionally substituted by a C₁₋₃-alkyl, amino-C₁₋₃-alkyl, C₁₋₃-alkylamino-C₁₋₃-alkyl, di-(C₁₋₃-alkyl)-amino-C₁₋₃-alkyl or phenyl-C₁₋₃-alkyl group or an oxygen or sulphur atom and additionally a nitrogen atom or
an imino group optionally substituted by a C₁₋₃-alkyl or phenyl-C₁₋₃-alkyl group and two or three nitrogen atoms,
and moreover a phenyl ring may be fused to the above-mentioned monocyclic heteroaryl groups via two adjacent carbon atoms
and the bond is effected via a nitrogen atom or a carbon atom of the heterocyclic moiety or a fused-on phenyl ring,
while the alkyl and alkoxy groups contained in the definitions which have more than two carbon atoms may, unless otherwise stated, be straight-chain or branched,
and the hydrogen atoms of the methyl or ethyl groups contained in the definitions may be wholly or partly replaced by fluorine atoms,
the tautomers, the enantiomers, the diastereomers, the mixtures thereof, the prodrugs thereof and the salts thereof.

2. The compound of general formula I according to claim 1, wherein
X denotes a nitrogen atom or a methyne group,
Y denotes a methyne group optionally by substituted a C₁₋₃-alkyl group,
Z denotes a nitrogen atom or a methyne group,
R¹ denotes an amino, C₁₋₅-alkylamino or C₃₋₇-cycloalkylamino group which may be substituted in each case at the amino nitrogen atom by a C₁₋₃-alkyl or C₁₋₃-alkyl-carbonyl group optionally substituted in the alkyl moiety by a carboxy group, a group which may be converted in vivo into a carboxy group, an amino, C₁₋₃-alkyl-amino or di-(C₁₋₃-alkyl)-amino group,
a 4- to 7-membered cycloalkyleneiminocarbonyl or cycloalkyleneiminosulphonyl group, while
the cycloalkyleneimino moiety may be substituted by a C₁₋₃-alkyl, amino-C₁₋₃-alkyl, C₁₋₃-alkylamino-C₁₋₃-alkyl, di-(C₁₋₃-alkyl)-amino-C₁₋₃-alkyl, aminocarbonyl, C₁₋₃-alkylamino-carbonyl, N—(C₃₋₇-cycloalkyl)-C₁₋₅-alkyl-aminocarbonyl or di-(C₁₋₃-alkyl)-aminocarbonyl group or
a methylene group not adjacent to the imino group may be substituted by a hydroxy, benzyloxy, amino, C₁₋₃-alkylamino or di-(C₁₋₃-alkyl)-amino group and/or
a methylene group in the 3 position of a 5-, 6- or 7-membered cycloalkyleneimino group may be replaced by a sulphur atom or by a sulphinyl or sulphonyl group or
a methylene group in the 4 position of a 6- or 7-membered cycloalkyleneimino group may be replaced by an oxygen or sulphur atom or by an —NH—, —N(C₂₋₃-alkanoyl)-, sulphinyl or sulphonyl group and/or
a —CH₂—CH₂— group in a 5- to 7-membered cycloalkyleneimino group may be replaced by a —NH—CO— group,
a 2,5-dihydropyrrol-1-yl-carbonyl or 1,2,5,6-tetrahydropyridin-1-yl-carbonyl group optionally substituted by a C₁₋₃-alkyl, amino-C₁₋₃-alkyl, C₁₋₃-alkylamino-C₁₋₃-alkyl, di-(C₁₋₃-alkyl)-amino-C₁₋₃-alkyl, aminocarbonyl, C₁₋₃-alkylamino-carbonyl, N—(C₃₋₇-cycloalkyl)-C₁₋₅-alkylaminocarbonyl, N-(phenyl-C₁₋₃-alkyl)-C₁₋₅-alkylaminocarbonyl or di-(C₁₋₃-alkyl)-aminocarbonyl group,
an aminosulphonyl or aminocarbonyl group optionally substituted by one or two C₁₋₃-alkyl, phenyl-C₁₋₃-alkyl or C₃₋₇-cycloalkyl groups, while the substituents may be identical or different,
a straight-chain or branched C₁₋₅-alkylcarbonyl group or
a C₃₋₇-cycloalkyl-carbonyl group,
R² denotes a hydrogen, fluorine, chlorine or bromine atom,
a C₁₋₃-alkyl group wherein the hydrogen atoms may be wholly or partly replaced by fluorine atoms, or
a C₂₋₃-alkenyl, C₂₋₃-alkynyl, hydroxy, C₁₋₃-alkoxy or trifluoromethoxy group,
R³ denotes a hydrogen atom,
a straight-chain or branched C₁₋₆-alkyl group which is optionally substituted by a hydroxy, C₁₋₃-alkyloxy, carboxy, C₁₋₃-alkoxy-carbonyl, amino, C₁₋₃-alkylamino, di-(C₁₋₃-alkyl)-amino, C₁₋₃-alkyl-carbonylamino, C₁₋₅-alkoxy-carbonylamino or phenyl-C₁₋₃-alkoxy-carbonylamino group, or
a methyl group which is substituted
by a phenyl or heteroaryl group which are optionally substituted in each case by a hydroxy, C₁₋₄-alkyloxy, benzyloxy, hydroxycarbonyl-C₁₋₃-alkoxy, C₁₋₃-alkyloxy-carbonyl-C₁₋₃-alkyloxy, aminocarbonyl-C₁₋₃-alkyloxy, C₁₋₃-alkyl-aminocarbonyl-C₁₋₃-alkyloxy, di-(C₁₋₃-alkyl)-aminocarbonyl-C₁₋₃-alkyloxy, carboxy, C₁₋₃-alkyloxy-carbonyl group, and
Ar denotes a phenyl group substituted by the groups R⁴, R⁵ and R⁶, while
R⁴ denotes a cyano group,
an amidino group optionally substituted by one or two hydroxy, C₁₋₃-alkyl, C₁₋₈-alkyl-carbonyl, C₁₋₈-alkoxy-carbonyl or benzoyl groups, an amino-C₁₋₃-alkyl, C₁₋₃-alkylamino-C₁₋₃-alkyl or di-(C₁₋₃-alkyl)amino-C₁₋₃-alkyl group or
a group of formula 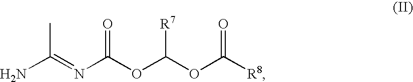
wherein R⁷ denotes a hydrogen atom or a C₁₋₃-alkyl group and
R⁸ denotes a C₁₋₃-alkyl group,
R⁵ denotes a hydrogen, fluorine, chlorine or bromine atom or a trifluoromethyl, C₁₋₃-alkyl, hydroxy, benzyloxy, amino or C₁₋₃-alkylamino group and
R⁶ denotes a hydrogen, chlorine or bromine atom or a C₁₋₃-alkyl group,
or a thienylene, thiazolylene, pyridinylene, pyrimidinylene, pyrazinylene or pyridazinylene group optionally substituted in the carbon skeleton by the groups R⁴ and R⁵, while R₄ and R₅ are as hereinbefore defined,
while, unless otherwise mentioned, by the term a “heteroaryl group” is meant a monocyclic 5- or 6-membered heteroaryl group optionally substituted in the carbon skeleton by a C₁₋₃-alkyl, carboxy, C₁₋₃-alkoxy-carbonyl or C₁₋₃-alkoxy-carbonylamino group, while
the 6-membered heteroaryl group contains one, two or three nitrogen atoms and
the 5-membered heteroaryl group contains an imino group optionally substituted by a C₁₋₃-alkyl or phenyl-C₁₋₃-alkyl group, or an oxygen or sulphur atom or
an imino group optionally substituted by a C₁₋₃-alkyl, amino-C₁₋₃-alkyl, C₁₋₃-alkylamino-C₁₋₃-alkyl, di-(C₁₋₃-alkyl)-amino-C₁₋₃-alkyl or phenyl-C₁₋₃-alkyl group, or an oxygen or sulphur atom and additionally contains a nitrogen atom or
an imino group optionally substituted by a C₁₋₃-alkyl or phenyl-C₁₋₃-alkyl group and two or three nitrogen atoms,
and moreover a phenyl ring may be fused to the above-mentioned monocyclic heteroaryl groups via two adjacent carbon atoms
and the bond is effected via a nitrogen atom or a carbon atom of the heterocyclic moiety or a fused-on phenyl ring,
while the alkyl and alkoxy groups contained in the definitions which have more than two carbon atoms may, unless otherwise stated, be straight-chain or branched,
and the hydrogen atoms of the methyl or ethyl groups contained in the definitions may be wholly or partly replaced by fluorine atoms,
the tautomers, the enantiomers, the diastereomers, the mixtures thereof, the prodrugs thereof and the salts thereof.

3. The compound of general formula I according to claim 2, wherein
X denotes a nitrogen atom or a methyne group,
Y denotes a methyne group and
Z denotes a nitrogen atom or a methyne group,
R¹ denotes an amino, C₁₋₅-alkylamino or C₃₋₇-cycloalkylamino group which may be substituted in each case at the amino nitrogen atom by a C₁₋₃-alkyl or C₁₋₃-alkyl-carbonyl group optionally substituted in the alkyl moiety by a carboxy group, a group which may be converted in vivo into a carboxy group, an amino, C₁₋₃-alkyl-amino or di-(C₁₋₃-alkyl)-amino group,
a 4- to 7-membered cycloalkyleneiminocarbonyl group, while
the cycloalkyleneimino moiety may be substituted by a C₁₋₃-alkyl, amino-C₁₋₃-alkyl, C₁₋₃-alkylamino-C₁₋₃-alkyl, di-(C₁₋₃-alkyl)-amino-C₁₋₃-alkyl, aminocarbonyl, C₁₋₃-alkylamino-carbonyl or di-(C₁₋₃-alkyl)-aminocarbonyl group or
a methylene group not adjacent to the imino group may be substituted by a hydroxy, benzyloxy, amino, C₁₋₃-alkylamino or di-(C₁₋₃-alkyl)-amino group and/or
a methylene group in the 3 position of a 5-, 6- or 7-membered cycloalkyleneimino group may be replaced by a sulphur atom or by a sulphinyl or sulphonyl group or
a methylene group in the 4 position of a 6- or 7-membered cycloalkyleneimino group may be replaced by an oxygen or sulphur atom or by an —NH—, —N(C₂₋₃-alkanoyl)-, sulphinyl or sulphonyl group and/or
a —CH₂—CH₂— group in a 5- to 7-membered cycloalkyleneimino group may be replaced by a —NH—CO— group,
a 2,5-dihydropyrrol-1-yl-carbonyl or 1,2,5,6-tetrahydropyridin-1-yl-carbonyl group optionally substituted by a C₁₋₃-alkyl, amino-C₁₋₃-alkyl, C₁₋₃-alkylamino-C₁₋₃-alkyl, di-(C₁₋₃-alkyl)-amino-C₁₋₃-alkyl, aminocarbonyl, C₁₋₃-alkylamino-carbonyl, N-(C₃₋₇-cycloalkyl)-C₁₋₅-alkylaminocarbonyl, N-(phenyl-C₁₋₃-alkyl)-C₁₋₅-alkylaminocarbonyl or di-(C₁₋₃-alkyl)-aminocarbonyl group,
an aminocarbonyl group optionally substituted by one or two C₁₋₃-alkyl or C₃₋₇-cycloalkyl groups, while the substituents may be identical or different,
a straight-chain or branched C₁₋₅-alkylcarbonyl group or
a C₃₋₇-cycloalkyl-carbonyl group,
R² denotes a hydrogen, fluorine, chlorine or bromine atom,
a C₁₋₃-alkyl group wherein the hydrogen atoms may be wholly or partly replaced by fluorine atoms, or
a C₁₋₃-alkoxy or trifluoromethoxy group,
R³ denotes a hydrogen atom,
a straight-chain or branched C₁₋₆-alkyl group which is optionally substituted by a hydroxy, C₁₋₃-alkyloxy, carboxy or C₁₋₃-alkoxy-carbonyl group, or
a methyl group which is substituted by a phenyl group, and
Ar denotes a phenyl group substituted by the groups R⁴ and R⁵, where
R⁴ denotes a cyano group,
an amidino group optionally substituted by one or two hydroxy, C₁₋₈-alkyl-carbonyl, C₁₋₈-alkoxy-carbonyl or benzoyl groups,
an amino-C₁₋₃-alkyl group or
a group of formula 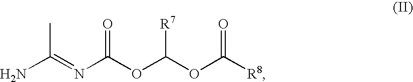
wherein R⁷ denotes a hydrogen atom or a C₁₋₃-alkyl group and
R⁸ denotes a C₁₋₃-alkyl group,
and R⁵ denotes a hydrogen atom or a hydroxy group,
or a thienylene group optionally substituted in the carbon skeleton by the group R₄, where R₄ is as hereinbefore defined,
while the alkyl and alkoxy groups contained in the definitions which have more than two carbon atoms may, unless otherwise stated, be straight-chain or branched,
and the hydrogen atoms of the methyl or ethyl groups contained in the definitions may be wholly or partly replaced by fluorine atoms,
the tautomers, the enantiomers, the diastereomers, the mixtures thereof and the salts thereof.

4. The compound of general formula I according to claim 3, wherein
X, Y, Z, R², R³ and Ar are defined as in claim 3 and
R¹ denotes a C₃₋₇-cycloalkylamino group which is substituted at the amino nitrogen atom by a C₁₋₃-alkyl-carbonyl group,
an azetidin-1-ylcarbonyl group optionally substituted in the 3 position by a dimethylamino group,
a pyrrolidin-1-yl-carbonyl or piperidin1-ylcarbonyl group optionally substituted in the 2 position by an aminomethyl or in the 3 position by an amino, aminomethyl, hydroxy or benzyloxy group,
a 2,5-dihydropyrrol-1-yl-carbonyl or thiazolidin-3-yl-carbonyl group or
a di-(C₁₋₃-alkyl)-aminocarbonyl or N-cyclohexyl-N—(C₁₋₃-alkyl)-aminocarbonyl group,
the tautomers, the enantiomers, the diastereomers, the mixtures thereof and the salts thereof.

5. The compound of general formula 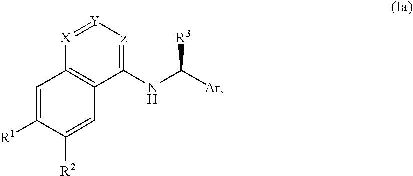
wherein X, Y, Z, R¹, R², R³ and Ar are defined as in claim 3,
the tautomers, the diastereomers, the mixtures thereof and the salts thereof.

6. The compound of general formula 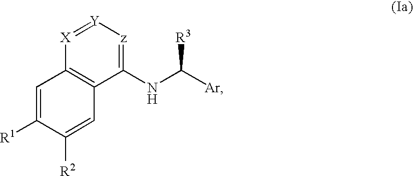
wherein X, Y, Z, R¹, R², R³ and Ar are defined as in claim 4,
the tautomers, the diastereomers, the mixtures thereof and the salts thereof.

7. The following compounds of general formula I according to claim 1:
(1) 3-{[7-(2,5-dihydropyrrol-1-yl-carbonyl)-quinazolin-4-yl]aminomethyl}-4-hydroxy-benzamidine 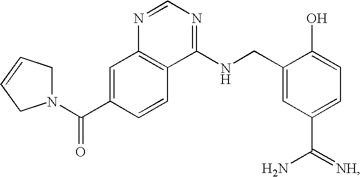
(2) 3-{2-phenyl-1-[7-(pyrrolidin-1-yl-carbonyl)-quinolin-4-ylamino]-ethyl}-benzamidine 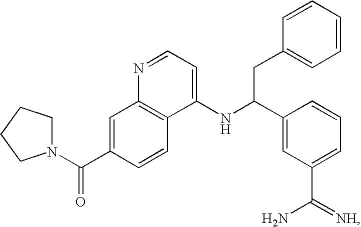
(3) 4-hydroxy-3-{[7-methoxy-6-(pyrrolidin-1-yl-carbonyl)-isoquinolin-1-yl]amino-methyl}-benzamidine 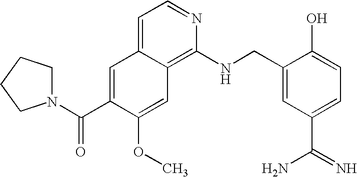
(4) 4-hydroxy-3-{2-phenyl-1-[7-(pyrrolidin-1-yl-carbonyl)-quinazolin-4-ylamino]-ethyl}-benzamidine 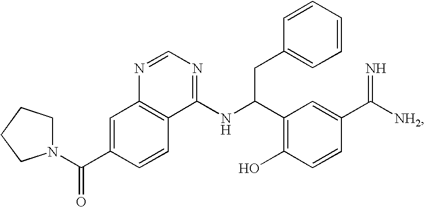
(5) 4-hydroxy-3-{[6-methyl-7-(pyrrolidin-1-yl-carbonyl)-quinazolin-4-yl]aminomethyl}-benzamidine 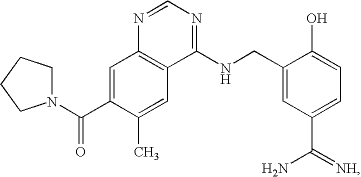
(6) ethyl 3-(3-amidino-phenyl)-3-{[6-chloro-7-(pyrrolidin-1-yl-carbonyl)-quinazolin-4-yl]amino}-propionate 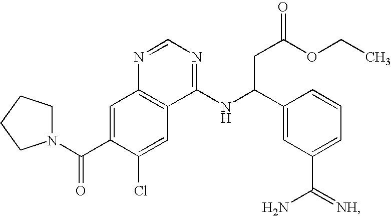
(7) 3-{[6-(N-acetyl-N-cyclopentylamino)-7-methyl-isoquinolin-1-yl]aminomethyl}-4-hydroxy-benzamidine 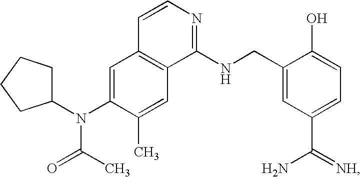
(8) N-acetoxymethoxycarbonyl-4-hydroxy-3-{[6-methyl-7-(pyrrolidin-1-yl-carbonyl)-quinazolin-4-yl]aminomethyl}-benzamidine 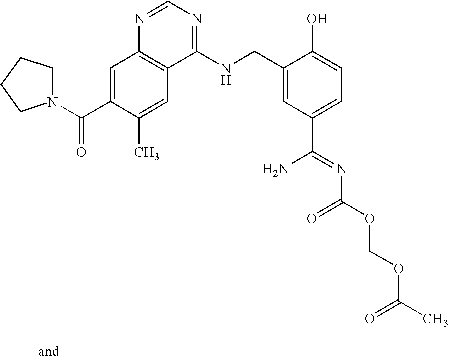
(9) 4-hydroxy-3-{[6-chloro-7-(pyrrolidin-1-yl-carbonyl)-quinazolin-4-yl]aminomethyl}-benzamidine 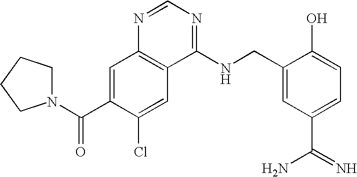
as well as the tautomers and the salts thereof.

8. A physiologically acceptable salt of the compound according to any one of claims 1 to 7, wherein R⁴ does not denote a cyano group.

9. A pharmaceutical composition containing at least one compound according to any one of claims 1 to 7, wherein R⁴ does not denote a cyano group, optionally together with one or more inert carriers and/or diluents.

10. A pharmaceutical composition containing a physiologically acceptable salt according to claim 8, optionally together with one or more inert carriers and/or diluents.

11. A process for preparing a pharmaceutical composition according to claim 9, wherein the compound is incorporated in one or more inert carriers and/or diluents by a non-chemical method.

12. A process for preparing a pharmaceutical composition according to claim 10, wherein the compound is incorporated in one or more inert carriers and/or diluents by a non-chemical method.

13. A process for preparing the compound of formula I according to any one of claims 1 to 7, comprising reacting
a compound of general formula 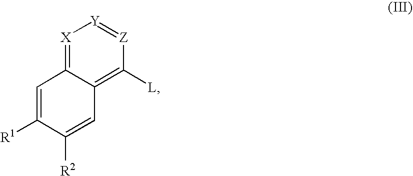
wherein
R¹, R², X, Y and Z are defined as in claims 1 to 7 and L denotes a leaving group such as a halogen atom, a sulphonyloxy or aryloxy group,
with a compound of general formula 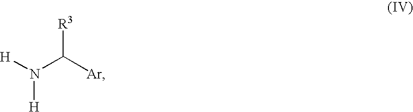
wherein Ar and R³ are defined as in claims 1 to 7,
and, if Ar is substituted by a cyano group, then the resulting cyano compound is optionally converted into one of the optionally substituted amidino or aminoalkyl compounds mentioned in claims 1 to 7 and
if desired any protective group used during the reactions to protect reactive groups is then cleaved, and/or
a compound of general formula I thus obtained is resolved into its stereoisomers, and/or
a compound of general formula I thus obtained is converted into the salts thereof, particularly for pharmaceutical use into the physiologically acceptable salts thereof with an inorganic or organic acid or base.
